(19) United States
(12) Patent Application Publication
(10) Pub. No.: US 2026/0224634 A1
(43) Pub. Date: Aug. 6, 2026

(54) Potency Assay and Manufacturing Method

(71) Applicant: MESOBLAST INTERNATIONAL SARL, Meyrin (CH)

(72) Inventors: Silviu Itescu, Melbourne, Victoria (AU); Jack Hayes, New York, NY (US)

(21) Appl. No.: 19/154,232

(22) PCT Filed: Feb. 8, 2024
(86) PCT No.: PCT/US2024/015019

§ 371 Date: Aug. 6, 2025

Related U.S. Application Data
(60) Provisional application No. 63/444,198, filed on Feb. 8, 2023.
(60) Provisional application No. 63/445,588, filed on Feb. 14, 2023.

(51) Int. Cl. A61K 35/28; A61P 37/06; C12N 5/0775; C12N 5/078; G01N 33/68

(52) U.S. Cl. CPC A61K 35/28; A61P 37/06; C12N 5/0634; C12N 5/0663; G01N 33/6869; C12N 2501/51; C12N 2501/515; C12N 2502/11; G01N 2333/7155

(57) ABSTRACT

The present disclosure relates to improved cellular compositions and potency assays for obtaining the same. Such compositions and assays may be suitable for use in the context of Tcell mediated disorders such as graft versus host disease (GvHD).

### FIELD OF THE DISCLOSURE

The present disclosure relates to improved cellular compositions and potency assays for obtaining the same. Such compositions and assays may be suitable for use in the context of T-cell mediated disorders such as graft versus host disease (GvHD).

### BACKGROUND

Several cellular therapy products for regenerative or immune therapy applications have advanced to clinical evaluation and market authorization. However, release of these cellular therapy products onto the market is hindered by their complexity and heterogeneity, which makes identification of relevant biologic activities and thus, definition of consistent cellular therapy product quality difficult.

Physiochemical parameters (for example, characterization of size, morphology, light-scattering properties, tensile strength, cell number, confluence, identification of phenotypic markers, secreted substances, genotype, gene expression profile) are routinely used for identification and quantification of the active substance, intermediates, impurities and contaminants. However, physiochemical parameters cannot confirm that a product will be biologically active and potent (i.e., elicit the desired effect). In contrast, biologic characterization takes into account the effect of the product on biologic systems, either modelled in vitro or in vivo in animals and ultimately in the clinic.

There is a need to develop cell products for treatment of diseases, for example, where immunosuppression is desired. It is also preferable to identify parameters that are critical to the efficacy of cellular therapy products and to control them (e.g., via potency testing) such that products of consistent quality can be obtained.

### SUMMARY OF THE DISCLOSURE

The present disclosure encompasses the surprising findings that:
i) IL-2Rα inhibition by stem cells in vitro correlates with stem cell directed suppression of T-cell activation in vivo; and,
ii) IL-2Rα inhibition levels by stem cells in vitro is unexpectedly effective at identifying drug product that will enhance survival in patients with GvHD, in particular those patients with severe GvHD.
   One or both of these findings underpin use of IL-2Rα inhibition as a potency assay and, further, provide a reference level of inhibition which can be used as release criteria for drug product. Accordingly, in a first example, the present disclosure relates to a method of selecting a cell population for use in treatment of graft versus host disease (GvHD), the method comprising: (i) obtaining a population of mesenchymal lineage precursor or stem cells (MLPSCs); (ii) culturing the MLPSC population in a culture medium; (iii) determining under culture conditions the level of IL-2RA inhibition; and, (iv) selecting for use in therapy an MLPSC population that inhibits IL-2RA by at least 56% under culture conditions. In an example, the method comprises selecting an MLPSC population for use in therapy which inhibits IL-2RA by at least 60% under culture conditions. In an example, the method comprises selecting an MLPSC population for use in therapy which inhibits IL-2RA by at least 65% under culture conditions.

In an example, the method further comprises cryopreserving the selected cells.

In another example, the present disclosure relates to a method for determining therapeutic efficacy of a population of culture expanded mesenchymal lineage precursor or stem cells (MLPSCs): (i) obtaining a population of MLPSCs; (ii) culturing the cells in a culture medium; and (iii) determining under culture conditions the level of IL-2RA inhibition, wherein the method determines therapeutic efficacy for graft versus host disease (GvHD) and, wherein at least 56% IL-2RA inhibition under culture conditions is indicative of therapeutic efficacy. In another example, ≥60% IL-2RA inhibition under culture conditions is indicative of therapeutic efficacy. In another example, ≥65% IL-2RA inhibition under culture conditions is indicative of therapeutic efficacy. For example, 65.7% IL-2RA inhibition under culture conditions is indicative of therapeutic efficacy.

In another example, the present disclosure relates to a method of manufacturing drug product which comprises a population of MLPSCs, the method comprising: acquiring a determination of whether a test population of MLPSCs inhibits IL-2RA at a predetermined level under culture conditions, and processing at least a portion of the test population of MLPSCs as a drug product if the test population of MLPSCs inhibits IL-2RA by at least the predetermined level under culture conditions, thereby manufacturing the drug product; or discarding at least a portion of the test population of MLPSCs if the population of mesenchymal stem cells inhibits IL-2RA by less than the predetermined level under culture conditions, wherein the predetermined level is 56%. In an example, the predetermined level ≥60% under culture conditions. In an example, the predetermined level ≥65% under culture conditions.

In another example, the present disclosure relates to a method of treating a subject with graft versus host disease (GvHD), the method comprising administering to a subject in need thereof a composition comprising a culture expanded population of mesenchymal lineage precursor or stem cells (MLPSCs), wherein the MLPSCs inhibit IL-2RA by at least 56% under culture conditions. In another example, the culture expanded MLPSCs which comprise the administered composition inhibit IL-2RA by ≥60% under culture conditions. In another example, the culture expanded MLPSCs which comprise the administered composition inhibit IL-2RA by ≥65% under culture conditions.

In an example, the GvHD is acute GvHD. In another example, the GvHD is chronic GvHD. In an example, the GvHD is pediatric GvHD. In an example, the GvHD is refractory to steroid therapy. In an example, the GvHD is grade D GvHD.

In an example, treatment increases 100 day survival to greater than 60%, greater than 70%.

In an example, the present disclosure relates to a composition comprising a population of culture expanded MLPSCs, wherein the population of MLPSCs are selected based on a predetermined level of IL-2RA inhibition under culture conditions, wherein the predetermined level of IL-2RA inhibition is at least 56%. In an example, the predetermined level of IL-2RA inhibition is ≥60%. In an example, the predetermined level of IL-2RA inhibition is ≥65%.

In an example, the MLPSCs are mesenchymal stem cells. In an example, the MSCs are one or more or all of CD29+, CD54+, CD73+, CD90+, CD102+, CD105+, CD106+, CD166+, MHC1+. In an example, the MSCs are selected based on expression of one or more or all of CD29, CD54, CD73, CD90, CD102, CD105, CD106, CD166, MHC1.

In an example, the level of IL-2RA inhibition is ≥65%.

In an example, the present disclosure provides a method of manufacturing drug product which comprises a population of MLPSCs, the method comprising (i) acquiring a determination of whether a test population of MLPSCs inhibits IL-2RA at a predetermined level under culture conditions, wherein step (i) comprises co-culturing the test population of MLPSCs with CD3/CD28 activated PBMCs; and determining the level of IL-2RA inhibition relative to a control population of CD3/CD28 activated PBMCs in the absence of MLPSCs; wherein the predetermined level of IL-2RA inhibition is at least 56%.

In an example, the control population of CD3/CD28 activated PBMCs express at least 12,000 pg/ml IL-2RA. In an example, the control population of CD3/CD28 activated PBMCs express at least 11,000 pg/ml IL-2RA. In an example, the control population of CD3/CD28 activated PBMCs express at least 13,000 pg/ml IL-2RA. In an example, the control population of CD3/CD28 activated PBMCs express at least 10,000 pg/ml IL-2RA. In an example, the control population of CD3/CD28 activated PBMCs express at least 12811 pg/ml IL-2RA.

In an example, CD3/CD28 activation of PBMCs comprises stimulating PBMCs with an anti-CD3 antibody and an anti-CD28 antibody.

In an example, the PBMCs are co-cultured with MLPSCs at a ratio of 5 PBMC:1 MPLSC.

In an example, 1×10⁶ PBMCs are co-cultured with 2×10⁵ MLPSCs.

In an example, the PBMCs and MLPSCs are co-cultured for a period of about 72 hours.

In an example, the level of IL-2RA inhibition is determined by: (i)
measuring IL-2RA levels in a test population of MLPSCs co-cultured with CD3/CD28-activated PBMCs, a positive control population of CD3/CD28-activated PBMCs in the absence of MLPSCs, and a negative control population of un-activated PBMCs in the absence of MLPSCs; (ii) constructing a standard curve to determine IL-2RA levels in the positive control population, negative control population, and test population; and (iii) calculating % inhibition of IL-2RA levels on the positive control population by the test population of MLPSCs co-cultured with CD3/CD28-activated PBMCs, relative to the positive control CD3/CD28-activated PBMC population.

In an example, the IL-2RA levels are measured by enzyme-linked immunosorbent assay (ELISA).

In an example, the standard curve is generated using a four-parameter logistic (4-PL) curve fit.

### BRIEF DESCRIPTION OF ACCOMPANYING FIGURES

FIG. 1: Correlation of IL-2Rα Inhibition and Clinical Outcomes.

FIG. 2: Product Potency Correlated with Survival in High-Risk Patients.

FIG. 3: Product Potency and Changes in Inflammatory Biomarkers.

FIG. 4: Product Potency and Changes in Activated T Cells.

FIG. 5: Relationship Between PBMC Stimulation And IL-2Rα Inhibition By MSC Lots.

FIG. 6: Survivors in GvHD Clinical Trial Received MSC Product With Higher Mean % IL2R Inhibition.

FIG. 7: Maintenance of potency as measured by % IL2Rα inhibition on repeat testing after 8 years.

FIG. 8: Accurate measurement of % Inhibition Of IL-2Rα in Commercial Inventory requires Adequate PBMC Stimulation And Induced Levels Of IL-2Rα Expression.

### DETAILED DESCRIPTION

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

Unless specifically defined otherwise, all technical and scientific terms used herein shall be taken to have the same meaning as commonly understood by one of ordinary skill in the art (e.g., in cell culture, molecular genetics, stem cell therapy, immunology, immunohistochemistry, protein chemistry, and biochemistry).

Unless otherwise indicated, the surgical techniques utilized in the present disclosure are standard procedures, well known to those skilled in the art.

Methods of obtaining and enriching a population of mesenchymal lineage stem or precursor cells are known in the art. For example, enriched populations of mesenchymal lineage stem or precursor cells can be obtained by the use of flow cytometry and cell sorting procedures based on the use of cell surface markers that are expressed on mesenchymal lineage stem or precursor cells.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### Selected Definitions

Methods of the present disclosure are performed on populations of MLPSCs. Such methods may be performed on a population of MLPSCs that is representative of a larger MLPSC population (e.g. a test population). In an example, methods of the present disclosure are performed on at least a portion of the test population of MLPSCs. In an example, such methods comprise processing at least a portion of a test population of MLPSCs as a drug product if the test population of MLPSCs inhibits IL-2RA by at least a predetermined level under culture conditions. In an example, such methods can further comprise discarding at least a portion of the test population of MLPSCs if the population of MLPSCs inhibits IL-2RA by less than the predetermined level under culture conditions. In an example, the predetermined level is 56%. In an example, the predetermined level is ≥56%. In another example, the predetermined level is ≥60%.

Interleukin-2 receptor alpha chain (“IL-2RA”; “IL-2Rα” also called CD25) is a validated marker of T-cell activation. In certain embodiments methods of the disclosure measure capability for inhibition of T-cell activation. For the avoidance of doubt, the terms “IL-2RA”, “IL-2Rα”, “IL-2R Alpha”, and variations thereof, are used interchangeably herein.

The term “predetermined” level is used in the context of the present disclosure to refer to a therapeutically effective amount of IL-2Rα inhibition. In an example, the predetermined level is a clinically proven effective predetermined level. In an example, the level is clinically proven effective in GvHD. In another example, the predetermined level is predetermined by a regulatory authority such as the US Food and Drug Administration (FDA). In an example, the predetermined level corresponds with increased survival in patients with GvHD. In an example, the predetermined level corresponds with increased survival in patients with grade D GvHD.

In an example, the predetermined level is a “reference level of IL-2Rα inhibition”. In an example, the reference level of inhibition is a level of inhibition of an FDA approved MLPSC population (e.g., an FDA MSC population approved for treatment of GvHD). In an example, the reference level of IL-2Rα provides the criteria for selecting cell populations according to the present disclosure.

In an example, the level of IL-2RA inhibition is determined under culture conditions. The term “culture conditions” is used to refer to cells growing in culture. In an example, culture conditions refers to an actively dividing population of cells. Such cells may, in an example, be in exponential growth phase.

In an example, culture conditions encompass co-culture of an MLPSC population disclosed herein and a second cell population such as a population which comprises peripheral blood mononuclear cells (PBMC). In an example, co-culture comprises culturing an MLPSC population disclosed herein and a population of activated PBMC. For example, PBMC can be activated using anti-CD3 and anti-CD28 antibodies before co-culture with an MLSPC population disclosed herein.

In an example, “culture conditions” comprises co-culturing MLPSCs and activated PBMC at a ratio of about 1 MLPSC:2 activated PBMC, or less. For example, 1:3, 1:4, 1:5, 1:10, 1:20, 1:30, 1:40, 1:50, 1:60, 1:70 1:80, 1:90, or 1 MLPSC: 100 activated PBMC, or less. In this example, the level of IL-2RA inhibition is determined after about 30 to 84 hours of cell culture under culture conditions.

In an example, “culture conditions” comprises co-culturing MLPSCs and T cells at a ratio of about 1 MLPSC:2 T cells, or less. For example, 1:3, 1:4, 1:5, 1:10, 1:20, 1:30, 1:40, 1:50, 1:60, 1:70 1:80, 1:90, or 1 MLPSC: 100 T cells, or less. In this example, the level of IL-2RA inhibition is determined after about 30 to 84 hours of cell culture under culture conditions.

In an example, the level of a particular marker can be determined by taking a sample of cell culture media and measuring the level of marker in the sample. In another example, the level of a particular marker can be determined by taking a sample of cells and measuring the level of the marker in the cell lysate. Those of skill in the art will appreciate that secreted markers can be measured by sampling the culture media while markers expressed on the surface of the cell may be measured by assessing a sample of cell lysate. In an example, the sample is taken when the cells are in exponential growth phase. In an example, the sample is taken after at least two or three days in culture. In another example, the sample is taken after about 30 to 84 hours of co-culture. In an example, the sample is taken from a co-culture of MLPSCs and activated PBMCs. In this example, the cell sample can be lysed and the level of a marker can be determined.

In an example, the level of IL-2RA may be determined using various methods such as an enzyme-linked immunosorbent assay (ELISA) based method. Suitable ELISA kits are known in the art and include, for example, the Quantikine Human CD25/IL-2 Rα ELISA. In an example, the ELISA comprises:
(i) adding sample diluent to each well of a microplate precoated with a monoclonal antibody specific for IL-2RA;
(ii) adding a co-cultured sample to a well of a microplate precoated with a monoclonal antibody specific for IL-2RA;
(iii) incubating the microplate for sufficient time to allow for the monoclonal antibody specific for IL-2RA to specifically bind to any IL-2RA in the sample;
(iv) washing the microplate;
(v) adding IL-2RA conjugate to the well;
(vi) incubating the microplate for sufficient time to allow the conjugate to specifically bind to any captured IL-2RA;
(vii) washing the microplate;
(viii) adding a substrate solution to the well;
(ix) incubating the microplate for sufficient time for colour development;
(x) adding a stop solution to the well;
(xi) reading optical density on a microplate reader set to 450 nm with wavelength correction at 570 nm;
(xii) determining the level of IL-2RA.

In another example, the level of IL-2RA is determined using fluorescence-activated cell sorting (FACS) using appropriate antibodies such as anti-CD25. Further antibodies may also be employed if required to distinguish CD25+ cell types.

In an example, the level of IL-2RA in co-culture is compared with the level of IL-2RA in cultured population of activated PBMC. Levels of IL-2RA are subsequently compared to provide a level of IL-2RA inhibition in co-culture. For example, the level of IL-2RA may be inhibited at least 60% in co-culture (i.e. MLPSC:activated PBMC) relative single culture (activated PBMC alone). Those of skill in the art will appreciate other suitable methods of determining level of IL-2RA under culture conditions.

In an example, the level of IL-2RA is determined by a standard curve. A “standard curve” refers to a general method for determining the concentration of a substance in an unknown sample by comparing the unknown to a set of standard samples of known concentrations. In an example, the standard curve is constructed using a four-parameter logistic (4-PL) curve fit. As the person skilled in the art would understand, four parameter logistic (4PL) curve is a regression model often used to analyze bioassays such as ELISA. They follow a sigmoidal, or “s”, shaped curve. This type of curve is particularly useful for characterizing bioassays because bioassays are often only linear across a specific range of concentration magnitudes.

Culture expanding cells from a cryopreserved intermediate means thawing cells subject to cryogenic freezing and in vitro culturing under conditions suitable for growth of the cells.

In an example, the “level” or “amount” of a particular marker such as IL-2RA is determined before cells have been cryopreserved. In another example, the level or amount of a particular marker is determined after a first cryopreservation of cells. In another example, the level is determined after a second cryopreservation of cells. For example, cells may be culture expanded to provide an intermediate, cryopreserved, defrosted before being re-seeded in culture and further culture expanded before the level of a particular marker can be determined under culture conditions.

The term “intermediate” is used in the context of the present disclosure to refer to an intermediate population of MLPSCs that have been freshly isolated and subject to early stage culture expansion (e.g. around 2 passages). For example, production of the intermediate population of MLPSCs can include the isolation of nucleated bone marrow cells (NBMC) from Bone Marrow Aspirate (BMA), cell culture and two passages to expand the relevant cells. In an example, cells are then harvested and formulated for analysis according to the methods described herein. In an example the harvested cells are cryopreserved. Accordingly, production of an intermediate population, is the first stage of the process of producing a composition for administration to a subject (i.e. drug product). While the above referenced example refers to isolation of NBMC from BMA, those of skill in the art will appreciate that relevant cells can be obtained from other suitable source(s) as discussed herein (e.g. adipose tissue) and subjected to the necessary culture expansion to intermediate and, drug product.

In certain examples disclosed herein, intermediates of the disclosure are culture expanded to provide a population of MLPSCs for administration. In an example, such populations can be referred to as a pharmaceutical composition or drug product (DP). For example, culture expansion of the intermediate involves cell culture and more passages (e.g. three more passages) for culture expansion.

By “isolated” or “purified” it is meant a cell which has been separated from at least some components of its natural environment. This term includes gross physical separation of the cells from its natural environment (e.g. removal from a donor). The term “isolated” includes alteration of the cell's relationship with the neighboring cells with which it is in direct by, for example, dissociation. The term “isolated” does not refer to a cell which is in a tissue section. When used to refer to the population of cells, the term “isolated” includes populations of cells which result from proliferation of the isolated cells of the disclosure.

The terms “passage”, “passaging” or “sub-culture” are used in the context of the present disclosure to refer to known cell culture techniques that are used to keep cells alive and growing under cultured conditions for extended periods of time so that cell numbers can continually increase. The degree of sub-culturing a cell line has undergone is often expressed as “passage number,” which is generally used to refer to the number of times cells have been sub-cultured. In an example, one passage comprises removing non-adherent cells and leaving adherent mesenchymal lineage precursor or stem cells. Such mesenchymal lineage precursor or stem cells can then be dissociated from the substrate or flask (e.g., by using a protease such as trypsin or collagenase), media can be added, optional washing (e.g., by centrifugation) may be performed, and then the mesenchymal lineage precursor or stem cells can be re-plated or reseeded to one or more culture vessels containing a greater surface area in total. The mesenchymal lineage precursor or stem cells can then continue to expand in culture. In another example, methods of removing non-adherent cells include steps of non-enzymatic treatment (e.g., with EDTA). In an example, mesenchymal lineage precursor or stem cells are passaged at or near confluence (e.g., about 75% to about 95% confluence). In an example, the mesenchymal lineage precursor or stem cells are seeded at a concentration of about 10%, about 15%, or about 20% cells/ml of culture medium.

The term “medium” or “media” as used in the context of the present disclosure, includes the components of the environment surrounding cells in culture. It is envisaged that the media contributes to and/or provides the conditions suitable to allow cells to grow. Media may be solid, liquid, gaseous or a mixture of phases and materials. Media can include liquid growth media as well as liquid media that do not sustain cell growth. Exemplary gaseous media include the gaseous phase that cells growing on a petri dish or other solid or semisolid support are exposed to.

As used herein, the terms “treating”, “treat” or “treatment” include administering a population of mesenchymal lineage stem or precursor cells and/or progeny thereof and/or soluble factors derived therefrom to thereby reduce or eliminate at least one symptom of GvHD. In an example, treatment includes administering a population of culture expanded mesenchymal lineage stem or precursor cells disclosed herein. In an example, treatment response is determined relative to baseline. In an example, treatment response is determined relative to a control patient population.

In an example, methods of the present disclosure inhibit disease progression or disease complication in a subject. “Inhibition” of disease progression or disease complication in a subject means preventing or reducing the disease progression and/or disease complication in the subject. Accordingly, in an example, methods of the disclosure inhibit progression of disease severity. For example, such methods can inhibit progression of GvHD severity (i.e. inhibit progression in grading).

The term “prevent” or “preventing” as used herein include administering a population of mesenchymal lineage stem or precursor cells and/or progeny thereof and/or soluble factors derived therefrom to thereby stop or inhibit the development of at least one symptom of inflammatory disease.

The term “subject” as used herein refers to a human subject. For example, the subject can be an adult. In another example, the subject is a pediatric subject. Terms such as “subject”, “patient” or “individual” are terms that can, in context, be used interchangeably in the present disclosure. In an example, the subject has GvHD.

As used herein, the term “genetically unmodified” refers to cells that have not been modified by transfection with a nucleic acid. For the avoidance of doubt, in the context of the present disclosure a mesenchymal lineage precursor or stem cell transfected with a nucleic acid encoding Ang1 would be considered genetically modified.

The term “clinically proven” (used independently or to modify the term “effective”) shall mean that efficacy has been proven by a clinical trial wherein the clinical trial has met the approval standards of U.S. Food and Drug Administration, EMEA or a corresponding national regulatory agency. For example, the clinical study may be an adequately sized, randomized, double-blinded study used to clinically prove the effects of the composition. In an example, a clinically proven effective amount is an amount shown by a clinical trial to meet a specified endpoint. In an example, the end point is protection against death. Put another way, the end point increases survival. For example, 100 day survival may be increased when administering cell populations meeting release criteria according to the present disclosure.

Accordingly, the terms “clinically proven efficacy” and “clinically proven effective” can be used in the context of the present disclosure to refer to a dose, dosage regimen, treatment or method disclosed herein. Efficacy can be measured based on change in the course of the disease in response to administering a composition disclosed herein. For example, a composition of the disclosure is administered to a subject in an amount and for a time sufficient to induce an improvement, preferably a sustained improvement, in at least one indicator that reflects the severity of inflammatory disease. Various indicators that reflect the severity of the disease can be assessed for determining whether the amount and time of the treatment is sufficient. Such indicators include, for example, clinically recognized indicators of disease severity or symptoms. In an example, the degree of improvement is determined by a physician, who can make this determination based on signs, symptoms, or other test results. In an example, a clinically proven effective amount improves patient survival. In another example, a clinically proven effective amount reduces a subjects risk of mortality. In another example, a clinically proven effective amount increases 100 day survival. In an example, methods of the disclosure administer a clinically proven effective amount of a composition disclosed herein.

“Therapeutic efficacy” is used in the context of the present disclosure to refer to MLPSCs and compositions disclosed herein that can treat, inhibit and/or prevent disease. For example, therapeutically effective MLPSCs and compositions disclosed herein can treat, inhibit and/or prevent an inflammatory disease such as GvHD. In an example, therapeutically effective MLPSCs and compositions disclosed herein increase 100 day survival in subjects with GvHD.

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the singular form “a”, “an” and “the” include singular and plural references unless the context indicates otherwise.

### Graft Versus Host Disease (GvHD)

In an example, the methods of the present disclosure relate to the provision of cell populations effective for treating graft versus host disease (GvHD). “Graft versus Host Disease (GvHD)” is an immunological disorder that is the major factor that limits the success and availability of allogeneic bone marrow or stem cell transplantation. GvHD occurs in acute (aGvHD) or chronic (cGvHD) forms. Acute GvHD usually manifests within 100 days following bone marrow or stem cell transplantation. Chronic GvHD generally manifests later than aGvHD (>100 days post transplantation) and has some features of autoimmune diseases. It may develop either de novo, following resolution of aGvHD or as an extension of aGvHD. Chronic GvHD can cause multiple, often debilitating symptoms, including widespread skin rashes, painful mouth ulcers, shortness of breath, and limb and joint pain. In an example, patients with cGvHD have impaired reconstitution of CD5+ B cells. In an example, cGvHD is refractory to steroid therapy. In an example, cGvHD is refractory to a biologic therapy. In an example cGvHD is refractory to steroid therapy and a biologic therapy.

GvHD severity can be graded by patterns of organ involvement and clinical performance status. Multi-organ involvement includes skin rash, liver involvement, and/or gastrointestinal (GI) involvement. Examples of skin rash, liver involvement, and GI involvement are provided in Table 1 and Table 2. In an example, the subject has GvHD with multi-organ involvement. In an example, subjects treated according to the disclosure have severe GvHD. In an example, severe GvHD is graded according to the Glucksberg scale (Glucksberg et al, 1974; Thomas et al, 1975) (Table 1). For example, the subject can have Grade II GvHD or Grade III/IV GvHD according to the Glucksberg scale. In one example, the subject has Grade II GvHD. In another example, the subject has Grade III/IV GvHD.

In another example, severe GvHD is graded according to IBMTR Severity Index (Table 2) (Rowlings et al., 1997). In one example, the subject has Grade B, Grade C, or Grade D GvHD according to the IBMTR severity scale. In an example, subject has Grade D GvHD.

In another example, the subject has Minnesota high risk GvHD. Minnesota high risk acute GvHD is defined as either skin stage 4; lower gastrointestinal (GI) stage 3-4 or liver stage 3-4; or skin stage 3+ and either lower GI 2-4 or liver stage 2-4 GvHD (MacMillan et al., 2015). In each of these examples, the subject can also have a high MAP score. For example, the subject can have a MAP score ≥0.29:

TABLE 1
Glucksberg clinical stage and grade of
acute GvHD (Rowlings et al., 1997).

| Stage | Skin | Liver | Intestinal tract |
|---|---|---|---|
| 1 | Maculopapular | Bilirubin 34-50 | >500 ml |
| | rash <25% of body | μmol/l | diarrhoea/d |
| | surface | | |
| 2 | Maculopapular | Bilirubin 51-102 | >1000 ml |
| | rash 25-50% of | μmol/l | diarrhoea/d |
| | body surface | | |
| 3 | Generalised | Bilirubin 103-225 | >1500 ml |
| | erythroderma | μmol/l | diarrhoea/d |
| 4 | Generalised | Bilirubin >225 | Severe abdominal |
| | erythroderma with | μmol/l | pain, with or |
| | bullous formation | | without ileus |
| | and desquamation | | |

| Grade | Degree of organ involvement |
|---|---|
| I | Stage 1-2 skin rash: no gut involvement: no liver involvement: |
| | no decrease in clinical performance |
| II | Stage 1-3 skin rash: stage 1 gut involvement or stage 1 liver |
| | involvement (or both): mild decrease in clinical performance |
| III | Stage 2-3 skin rash: stage 2-3 gut involvement or stage 2-4 liver |
| | involvement (or both): marked decrease in clinical performance |
| IV | Similar to Grade III with stage-4 organ involvement and |
| | extreme decrease in clinical performance |

TABLE 2
Criteria for IBMTR Severity Index for
acute GvHD (Rowlings et al., 1997).

| | | | | Liver | | | Gastrointestinal | |
|---|---|---|---|---|---|---|---|---|
| | Skin | | | involvement | | | invovlement | |
| | involvement | | | | Total | | | Volume |
| | | Extent | | | bili- | | | of diar- |
| | Stage | of | | Stage | rubin | | Stage | rhoea |
| Index* | (max) | rash | | (max) | (μmol/l) | | (max) | (ml/d) |
| A | 1 | <25% | | 0 | <34 | | 0 | <500 |
| B | 2 | 25-50% | or | 1-2 | 34-102 | or | 1-2 | 550-1500 |
| C | 3 | >50% | or | 3 | 103-255 | or | 3 | >1500 |
| D | 4 | Bullae | or | 4 | >255 | or | 4 | Severe |
| | | | | | | | | pain and |
| | | | | | | | | ileus |

*Assign Index based on maximum in an individual organ system

In an example, subjects with severe GvHD do not respond to treatment with primary therapy. In an example, the subject has worsened within 3 days of primary therapy. In this example, the subject has worsened if their GvHD has increased in severity. For example, the subject's GvHD severity has increased according to MAP. In another example, the subject's GvHD severity has increased according to the Glucksberg scale. In another example, the subject's GvHD severity has increased according to the IMBTR scale. In another example, the subject's GvHD severity has increased according to organ involvement.

In another example, the subject has not responded within 7 days of a primary therapy. For example, the subject is refractory to primary therapy. In one example, primary therapy is systemic steroids. In one example, the subject has severe GvHD and is refractory to a steroid. In an example, the steroid is a corticosteroid. In another example, the steroid is a glucocorticoid. In another example, the steroid is prednisone. In another example, the subject has severe GvHD and is refractory to steroids and a second line therapy. For example, a second line therapy can include extracorporeal photopheresis, etanercept, infliximab, ruxolitinib, anti-thymocyte globulin, mycophenolate, alemtuzumab, basiliximab, or tocilizumab.

### Treatment Response

Methods of the present disclosure relate to the treatment of GvHD. As used herein, the terms “treating”, “treat”, “treatment”, “reducing progression” include administering a population of mesenchymal lineage stem or precursor cells and/or progeny thereof and/or soluble factors derived therefrom and/or extracellular vesicles derived therefrom to thereby reduce or eliminate at least one symptom of the GvHD.

As used herein, the term “response” means response to therapy. In an example, a subject is considered to have had a response if they have an improvement in at least one organ without progression in any other organs and if additional therapy was not required. In another example, a subject is considered not to have had a response if they had stable or progressive GvHD or if the subsequent addition of secondary therapy is required. In this example, a subject who does not have a response is a non-responder.

In an example, treatment induces a partial response. In an example, the partial response is induced at least 28 after treatment is initiated. In an example, the partial response is induced 28 days after treatment is initiated. In an example, the partial response is induced at least 30 days after treatment is initiated. In an example, the partial response is induced at least 2 months after treatment is initiated. In another example, the partial response is induced at least 3 months after treatment is initiated. In another example, the partial response is induced within 3 months. In another example, the partial response is induced 28 to 56 days after treatment is initiated. In another example, the partial response is induced 100 days after treatment is initiated. In another example, the partial response is induced 160 days after treatment is initiated. In another example, the partial response is induced 180 days after treatment is initiated.

In another example, the partial response is induced after two doses. In another example, the partial response is induced after two doses administered once weekly. In another example, the partial response is induced after two doses administered once weekly every two weeks. In another example, the partial response is induced after three doses or more. In an example, in the context of GvHD, a partial response is characterized by one or more or all of:
Reduction in Skin % BSA score of at least one point;
Reduction in mouth score of at least one point;
Reduction in eye score of at least one point;
Reduction in skin features score of at least one point;
Reduction in gastrointestinal tract score of at least one point;
Reduction in liver score of at least one point;
Reduction in lung symptom score of at least one point;
Reduction in lung FEV1 score of at least one point;
Reduction in joints and fascia score of at least one point;
Reduction in genital tract score of at least one point.

In an example, a partial response is characterized by a reduction in Skin % BSA score of at least one point. In another example, a partial response is characterized by a reduction in mouth score of at least one point. In another example, a partial response is characterized by a reduction in eye score of at least one point. In these examples, scores can be obtained using the NIH Consensus Criteria 2014 for GvHD.

In another example, a partial response is characterized by one or more or all of:
Reduction in Skin % BSA score of at least one point;
Reduction in mouth score of at least one point;
Reduction in eye score of at least one point.

There are various classification systems for characterizing GvHD (Lee, S., (2017) Blood., 129 (1): 30-37). In an example, the NIH Consensus Criteria 2014 can be used for scoring outcomes disclosed herein (Jagasia et al., (2015) Biol Blood Marrow Transplant., 21:389-401). The components of the NIH Consensus Criteria 2014 are shown in the following table:

TABLE 3
Organ Scoring of GvHD

| Organ | Score 0 | Score 1 | Score 2 | Score 3 |
|---|---|---|---|---|
| Skin % BSA¹ | No BSA | 1-18% BSA | 19-50% BSA | >50% BSA |
| | involved | | | |
| Skin Features | No sclerotic | N/A | Superficial sclerotic | Deep sclerotic features; |
| | features | | features, but not | “hidebound”; impaired |
| | | | “hidebound” | mobility; ulceration |
| Mouth | No | Mild | Moderate symptoms | Severe symptoms with |
| | symptoms | symptoms | with disease signs with | disease signs with |
| | | with disease | partial limitation of | major limitation of oral |
| | | signs but not | oral intake | intake |
| | | limiting oral | | |
| | | intake | | |
| | | significantly | | |
| Eyes | No | Mild dry eye | Moderate dry eye | Severe dry eye |
| | symptoms | symptoms | symptoms partially | symptoms significantly |
| | | not affecting | affecting ADL | affecting ADL (special |
| | | ADL | (requiring lubricant | eyewear to relieve |
| | | (requirement | drops >3x/day or | pain) OR unable to |
| | | of lubricant | punctal plugs) | work because of ocular |
| | | drops ≤3x/day) | WITHOUT new | symptoms OR loss of |
| | | | vision impairment due | vision due to |
| | | | to keratoconjunctivitis | keratoconjunctivitis |
| | | | sicca (KCS) | sicca (KCS) |
| GI Tract | No | Symptoms | Symptoms associated | Symptoms associated |
| | symptoms | without | with mild to moderate | with significant weight |
| | | significant | weight loss (5-15%) | loss (>15%) within 3 |
| | | weight loss | within 3 months OR | months, requires |
| | | (<5%) | moderate diarrhea | nutritional supplement |
| | | | without significant | for most calorie needs |
| | | | interference with daily | OR esophageal dilation |
| | | | living | OR severe diarrhea |
| | | | | with significant |
| | | | | interference with daily |
| | | | | living |
| Liver | Normal total | Normal total | Elevated total bilirubin | Elevated total bilirubin |
| | bilirubin and | bilirubin with | but ≤3 mg/dL or | but >3 mg/dL |
| | ALT or | ALT ≥3 to | ALT >5 × ULN | |
| | AP <3 × ULN | 5 × ULN or | | |
| | | AP ≥3 × | | |
| | | ULN | | |
| Lungs Symptom | No | Mild | Moderate symptoms | Severe symptoms |
| Score | symptoms | symptoms | (SOB after walking on | (SOB at rests; requires |
| | | (SOB after | flat ground) | O2) |
| | | climbing one | | |
| | | flight of | | |
| | | steps) | | |
| Lungs Lung Score | FEV1 ≥80% | FEV1 | FEV1 40-59% | FEV1 ≤39% |
| | | 60-79% | | |
| Joints and Fascia | No | Mild | Tightness of arms or | Contractures WITH |
| | symptoms | tightness of | legs OR joint | significant decrease of |
| | | arms and | contractures, erythema | range of motion AND |
| | | legs, normal | thought to be due to | significant limitation of |
| | | or mild | fasciitis, moderate | ADL (unable to tie |
| | | decreased | decrease of range of | shoes, button shirts, |
| | | range of | motion AND mild to | dress shelf, etc.) |
| | | motion AND | moderate limitation of | |
| | | not affecting | ADL | |
| | | ADL | | |
| Genital Tract | No signs | Mild signs | Moderate signs and | Severe signs with or |
| | | and females | may have signs of | without symptoms |
| | | with or | discomfort on exam | |
| | | without | | |
| | | discomfort | | |
| | | on exam | | |
| Other features | No GvHD | Mild | Moderate | Severe |

In an example, a partial response is a decrease of ≥1 point on the organ-specific NIH Consensus Criteria 2014 score from the Table above. Accordingly, in an example, treatment induces ≥1 point decrease in Skin % BSA score. In another example, treatment induces ≥1 point decrease in mouth score. In another example, treatment induces ≥1 point decrease in eye score. In another example, treatment induces ≥1 point decrease in skin features score. In another example, treatment induces ≥1 point decrease in gastrointestinal tract score. In another example, treatment induces ≥1 point decrease in liver score. In another example, treatment induces ≥1 point decrease in lung symptom score. In another example, treatment induces ≥1 point decrease in lung FEV1 score. In another example, treatment induces ≥1 point decrease in joints and fascia score. In another example, treatment induces ≥1 point decrease in genital tract score.

In an example, the treatment induces a complete response after treatment is initiated. In an example, a complete response is the complete resolution of GvHD symptoms in all organs. In an example, the complete response is induced 28 days after treatment is initiated. In an example, the complete response is induced at least 28 after treatment is initiated. In an example, the complete response is induced at least 30 after treatment is initiated. In an example, the complete response is induced at least 2 months after treatment is initiated. In another example, the complete response is induced at least 3 months after treatment is initiated. In another example, the complete response is induced 28 to 56 days after treatment is initiated. In another example, the complete response is induced 100 days after treatment is initiated. In another example, the complete response is induced 160 days after treatment is initiated. In another example, the complete response is induced 180 days after treatment is initiated.

In another example, the complete response is induced after two doses. In another example, the complete response is induced after two doses administered once weekly. In another example, the complete response is induced after two doses administered once weekly every two weeks. In another example, the complete response is induced after three doses or more.

In another example, again in the context of GvHD, the treatment increases the probability of the subject's survival. For example, treatment increases the probability of the subject surviving for at least 20 days to 200 days after initiation of treatment. In one example, treatment increases the probability of the subject surviving for at least 180 days after initiation of treatment. In another example, treatment increases the probability of the subject surviving at least 100 days. In an example, the increased probability is determined relative to a subject that is not treated with a composition of the disclosure. In an example, treatment increases 100 day survival greater than 60%. In another example, treatment increases 100 day survival greater than 60%. In another example, treatment increases 100 day survival greater than 65%. In another example, treatment increases 100 day survival greater than 68%. In another example, treatment increases 100 day survival greater than 70%. In another example, treatment increases 100 day survival greater than 71%. In another example, treatment increases 100 day survival greater than 72%. In another example, treatment increases 100 day survival between 60% and 75%. In another example, treatment increases 100 day survival between 65% and 75%. In another example, treatment increases 100 day survival between 70% and 75%. In an example, 100 day survival is determined across a population of patients with the average 100 day survival observed in the population providing the measure of 100 day survival for each patient in the population. In an example, this measure is compared relative to a subject (or population of subjects) that is not treated with a composition of the disclosure.

### Mesenchymal Lineage Precursor or Stem Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X % mesenchymal lineage precursor or stem cells”, wherein X % is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the mesenchymal lineage precursor or stem cells are CD29+, CD54+, CD73+, CD90+, CD102+, CD105+, CD106+, CD166+, MHC1+ MSCs.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+). For example, the MPCs can be isolated from bone mononuclear cells with an anti-STRO-3 antibody.

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+ (HSP-90B), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal lineage precursor or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1+ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

In an example, compositions of the disclosure comprise cells that induce new blood vessel formation in target tissue. In an example, the target tissue is the heart. In another example, the cells secrete factors that protect at risk or damaged myocardium. In an example, at risk or damaged myocardium has been subject to a lack of blood flow resulting from an ischemic event. In an example, the cells secrete factors that reduce apoptosis in cardiomyocytes.

### Culture Expansion of the Cells

In an example, mesenchymal lineage precursor or stem cells are culture expanded. “Culture expanded” mesenchymal lineage precursor or stem cells are distinguished from freshly isolated cells in that they have been cultured in cell culture medium and passaged (i.e. sub-cultured). In an example, freshly isolated cells are culture expanded for about 1 or 2 passages to provide an intermediate population. In an example, freshly isolated cells are culture expanded for 2 passages to provide an intermediate population. In another example, freshly isolated cells are culture expanded for about 1 to 3 passages to provide an intermediate population.

Accordingly, in an example, relevant cells are isolated and culture expanded for 2 passages to provide an intermediate MLPSC population. The intermediate MLPSC population is then culture expanded to provide a DP. For example, the intermediate cell population can be cultured for three more passages (i.e. 5 passages total) to provide a DP.

In an example, mesenchymal lineage precursor or stem cells are culture expanded for about 4-10 passages. In an example, mesenchymal lineage precursor or stem cells are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 7 passages. In these examples, MLPSCs may be culture expanded before being cryopreserved to provide an intermediate cryopreserved MLPSC population and then subject to further culture expansion.

In an example, DP compositions of the present disclosure are produced by culturing cells from an intermediate cryopreserved MLPSC population or, put another way, a cryopreserved intermediate.

In an example, compositions of the disclosure comprise mesenchymal lineage precursor or stem cells that are culture expanded from a cryopreserved intermediate. In an example, the cells culture expanded from a cryopreserved intermediate are culture expanded for at least 3, at least 5, at least 6, at least 7, at least 8, at least 9, at least 10 passages. For example, mesenchymal lineage precursor or stem cells can be culture expanded for at least 3 passages. In an example, mesenchymal lineage precursor or stem cells can be culture expanded for at least 3-10 passages. In an example, mesenchymal lineage precursor or stem cells can be culture expanded for at least 3-8 passages. In an example, mesenchymal lineage precursor or stem cells can be culture expanded for at least 3 7 passages.

In an example, IL-2RA inhibition by mesenchymal lineage precursor or stem cells culture expanded from a cryopreserved intermediate disclosed herein are assessed under culture conditions (e.g. co-culture with T-cells). In an example, these MLPSCs inhibit IL-2RA greater than 56% under culture conditions. In another example, these MLPSCs inhibit IL-2RA greater than 60% under culture conditions.

In an example, culture expanded mesenchymal lineage precursor or stem cells can be culture expanded in medium free of animal proteins (e.g. in a xeno-free medium). In an example, mesenchymal lineage precursor or stem cells can be culture expanded in medium that is fetal bovine serum free.

In an embodiment, mesenchymal lineage precursor or stem cells can be obtained from a single donor, or multiple donors where the donor samples or mesenchymal lineage precursor or stem cells are subsequently pooled and then culture expanded as required. In an example, the culture expansion process comprises:
i. expanding by passage expansion the number of viable cells to provide a preparation of at least about 1 billion of the viable cells, wherein the passage expansion comprises establishing a primary culture of isolated mesenchymal lineage precursor or stem cells and then serially establishing a first non-primary (P1) culture of isolated mesenchymal lineage precursor or stem cells from the previous culture;
ii. expanding by passage expansion the P1 culture of isolated mesenchymal lineage precursor or stem cells to a second non-primary (P2) culture of mesenchymal lineage precursor or stem cells; and,
iii. preparing and cryopreserving an in-process intermediate mesenchymal lineage precursor or stem cells preparation obtained from the P2 culture of mesenchymal lineage precursor or stem cells; and, optionally
iv. thawing the cryopreserved in-process intermediate mesenchymal lineage precursor or stem cells preparation and expanding by passage expansion the in-process intermediate mesenchymal lineage precursor or stem cells preparation.

In the context of the present disclosure, certain assays may be performed between steps iii and iv. For example, IL-2RA inhibition may be determined under culture conditions after step iii. In an example, step iv is only performed if a desired level of IL-2RA inhibition is observed under culture conditions. In this example, the cell population is selected for culture expansion on the basis of IL-2RA inhibition under culture conditions.

In an example, the expanded mesenchymal lineage precursor or stem cell preparation has an antigen profile and an activity profile comprising:
i. less than about 0.75% CD45+ cells;
ii. at least about 95% CD105+ cells;
iii. at least about 95% CD166+ cells.

In an example, the expanded mesenchymal lineage precursor or stem cell population is expanded from an intermediate MLPSC population and is capable of inhibiting IL-2RA expression by CD3/CD28-activated PBMCs by at least 56% relative to a control. In another example, the expanded mesenchymal lineage precursor or stem cell population is expanded from an intermediate MLPSC population and is capable of inhibiting IL-2RA expression by CD3/CD28-activated PBMCs by at least 60% relative to a control.

The process of mesenchymal lineage precursor or stem cell isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although mesenchymal lineage precursor or stem cells can generally withstand a certain amount of insult during preparation, cells are preferably manipulated by handling procedures and/or equipment that adequately performs the given step(s) while minimizing insult to the cells.

In an example, mesenchymal lineage precursor or stem cells are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a mesenchymal lineage precursor or stem cell composition cultured according to the present disclosure is 95% homogeneous with respect to being CD105 positive and CD166 positive and being CD45 negative. In an example, this homogeneity persists through ex vivo expansion; i.e. though multiple population doublings.

In an example, mesenchymal lineage precursor or stem cells of the disclosure are culture expanded in 2D culture. For example, mesenchymal lineage precursor or stem cells of the disclosure can be culture expanded in a cell factory. In certain examples, 3D culture of intermediates disclosed herein may follow using, for example, a bioreactor. In an example, mesenchymal lineage precursor or stem cells of the disclosure are initially culture expanded in 2D culture prior to being further expanded in 3D culture. In an example, intermediate cell populations of the disclosure have not been culture expanded in 3D culture. In an example, IL-2RA inhibition is assessed after culture expansion in 3D culture.

In an example, mesenchymal lineage precursor or stem cells of the disclosure are culture expanded from an intermediate population. In an example, mesenchymal lineage precursor or stem cells of the disclosure are culture expanded from the intermediate in 2D culture before seeding in 3D culture.

In the context of both intermediate populations and therapeutic compositions expanded from the same, in an example, mesenchymal lineage precursor or stem cells of the disclosure are culture expanded in 2D culture for at least 3 days before seeding in a further culture system such as cell factory or 3D culture in a bioreactor. In an example, mesenchymal lineage precursor or stem cells of the disclosure are culture expanded in 2D culture for at least 4 days before seeding in a further culture system. In an example, mesenchymal lineage precursor or stem cells of the disclosure are culture expanded in 2D culture for between 3 and 5 days before seeding in a further culture system. In these examples, 2D culture can be performed in a cell factory. Various cell factory products are available commercially (e.g. Thermofisher, Sigma, Corning). In an example, the cell factory has at least 5 layers. In an example, the cell factory has at least 10 layers. In an example, the cell factory has at least 20 layers. 3D culture may be performed in various bioreactor types such as stirred tank, wave bag, and vertical wheel.

In an example, CO₂ is provided during culture expansion of MLPSCs. In an example, MLPSCs are culture expanded in less than 9% CO₂. In an example, MLPSCs are culture expanded in less than 8% CO₂. In an example, MLPSCs are culture expanded in 5% CO₂. For example, MLPSCs can be culture expanded in 5%+/−2% CO₂. In an example, the MLPSCs are culture expanded with passive priming of CO₂. For example, cell factories can be passively primed with 5% CO₂.

Priming cell factories maintains the CO₂ tension between the cell factory and incubator and stabilizes the pH level of the growth medium. Active priming involves actively passing CO₂ gas through a bacterial vent air filter into each culture vessel (e.g. cell factory) for a defined period of time (e.g. around 10 minutes). However, active priming has the potential to introduce contamination into culture as it requires an open port to provide gas. Passive priming involves placing a closed culture system into an incubator at appropriate CO₂ concentration prior to cell seeding (e.g. around 12 to 72 hours).

In an example, cells of the disclosure are STRO-3+ before they are culture expanded to provide an intermediate cell population.

### Cell Culture Medium

Mesenchymal lineage precursor or stem cells disclosed herein can be culture expanded in various suitable growth mediums.

The term “medium” or “media” as used in the context of the present disclosure, includes the components of the environment surrounding the cells. The media contributes to and/or provides the conditions suitable to allow cells to grow. Media may be solid, liquid, gaseous or a mixture of phases and materials. Media can include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to.

The cell culture media used for culture expansion contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest. For example, it may be necessary to increase the level of glucose (or other energy source) in the basal medium, or to add glucose (or other energy source) during the course of culture, if this energy source is found to be depleted and to thus limit growth. In an example, dissolved oxygen (DO) levels can also be controlled.

In an example, the cell culture medium contains human derived additives. For example, human serum and human platelet cell lysate can be added to the cell culture media.

In an example, the cell culture medium contains only human derived additives. Thus, in an example, the cell culture media is xeno-free. For avoidance of doubt, in these examples, the culture medium is free of animal proteins. In an example, cell culture medium used in the methods of the disclosure is free of animal components.

In an example, the culture medium comprises serum. For example, fetal calf serum and/or new born calf serum. In other examples the culture medium is fetal bovine serum free culture medium comprising growth factors that promote mesenchymal lineage precursor or stem cell proliferation. In an embodiment, the culture medium is serum free stem cell culture medium. In an example, the cell culture medium comprises:
a basal medium;
platelet derived growth factor (PDGF);
fibroblast growth factor 2 (FGF2).

In an example, the culture medium comprises platelet derived growth factor (PDGF) and fibroblast growth factor 2 (FGF2), wherein the level of FGF2 is less than about 6 ng/ml. For example, the FGF2 level may be less than about 5 ng/ml, less than about 4 ng/ml, less than about 3 ng/ml, less than about 2 ng/ml, less than about 1 ng/ml. In an example, the FGF2 level is about 1 ng/ml.

In an example, the PDGF is PDGF-BB. In an example, the level of PDGF-BB is between about 1 ng/ml and 150 ng/ml. In another example, the level of PDGF-BB is between about 7.5 ng/ml and 120 ng/ml. In another example, the level of PDGF-BB is between about 15 ng/ml and 60 ng/ml. In another example, the level of PDGF-BB is about 10 ng/ml. In another example, the level of PDGF-BB is at least about 10 ng/ml or less.

In other examples, additional factors can be added to the cell culture medium. In an example, the culture medium further comprising EGF. EGF is a growth factor that stimulates cell proliferation by binding to its receptor EGFR. In an example, the method of the present disclosure comprises culturing a population of stem cells in a fetal bovine serum free cell culture medium further comprising EGF. In an example, the level of EGF is between about 0.1 and 7 ng/ml. For example, the level of EGF can be at least about 5 ng/ml.

In another example, the level of EGF is between about 1 ng/ml and 8 ng/ml. In another example, the level of EGF is between about 3 ng/ml and 6 ng/ml. In another example, the level of EGF is about 5 ng/ml. In another example, the level of EGF is about 5 ng/ml or less.

In the above examples, basal medium such as Alpha MEM or StemSpan™ can be supplemented with the referenced quantity of growth factor. In an example, the culture medium comprises Alpha MEM or StemSpan™ supplemented with 10 ng/ml PDGF-BB, 5 ng/ml EGF and 1 ng/ml FGF.

In other examples, additional factors can be added to the cell culture medium. For example, the cell culture media can be supplemented with one or more stimulatory factors selected from the group consisting of epidermal growth factor (EGF), 1α,25-dihydroxyvitamin D3 (1,25D), tumor necrosis factor α (TNF-α), interleukin-1β (IL-Iβ) and stromal derived factor 1α (SDF-1α). In another embodiment, cells may also be cultured in the presence of at least one cytokine in an amount adequate to support growth of the cells. In another embodiment, cells can be cultured in the presence of heparin or a derivative thereof. In an example, the heparin derivative is a sulphate). Various forms of heparin sulphate are known in the art and include heparin sulphate 2 (HS2). HS2 can be derived from various sources including for example, the liver of male and/or female mammals. Thus, an exemplary heparin sulphate includes male liver heparin sulphate (MML HS) and female liver heparin sulphate (FML HS).

In another example, the cell culture medium of the present disclosure maintain stem cells in an undifferentiated state. Stem cells are considered to be undifferentiated when they have not committed to a specific differentiation lineage. As discussed above, stem cells display morphological characteristics that distinguish them from differentiated cells. Furthermore, undifferentiated stem cells express genes that may be used as markers to detect differentiation status. The polypeptide products may also be used as markers to detect differentiation status. Accordingly, one of skill in the art could readily determine whether the methods of the present disclosure maintain stem cells in an undifferentiated state using routine morphological, genetic and/or proteomic analysis.

Co-Culture of Mesenchymal Lineage Precursor or Stem Cells with Stimulated or Activated T Cells

Interleukin-2 receptor alpha chain (“IL-2RA”; “IL-2Rα” also called CD25) is a validated marker of T-cell activation. In certain embodiments methods of the disclosure measure the inhibition of T-cell activation. In an example, T-cell activation is determined based on the level of T-cell IL-2RA expression following co-culture of activated PBMC with mesenchymal lineage precursor or stem cells. Inhibition of IL-2RA expression is associated with a suppressive effect on T cell activation.

In an example, activated PBMC are co-cultured with mesenchymal lineage precursor or stem cells in a culture medium comprising at least one T cell stimulating agent, preferably at a concentration capable of stimulating and/or activating the T cells. In another embodiment, the T cells are first stimulated and/or activated prior to co-culture with the mesenchymal lineage precursor or stem cells.

In an example, mesenchymal lineage precursor or stem cells are co-cultured with PBMC in a culture medium comprising an agent that can stimulate CD3 and an agent that can stimulate CD28 on T cells, such as an antibody to CD3 and an antibody to CD28, for example, mouse anti-human CD3 and mouse anti-human CD28. In an example, the antibody to CD3 and/or the antibody to CD28 is added to the culture medium in soluble form, each at a concentration of about 2 μg/ml.

In an embodiment, the PBMC are co-cultured with mesenchymal lineage precursor or stem cells at a ratio of 5 PBMC:1 mesenchymal lineage precursor or stem cell. For example, 1×10⁶ PBMCs could be co-cultured with 2×10⁵ MLPSCs. In an example, cells can be co-cultured in a final volume of 1 ml.

In an example, MLPSCs are co-cultured with anti-CD3/CD28 antibody-stimulated PBMCs and IL-2Rα expression by activated PBMCs is measured by enzyme-linked immunosorbent assay (ELISA).

In an example, level of IL-2Rα is determined relative to a control such as level of IL-2Rα expression by activated PBMCs in the absence of MLPSCs.

### Determining Therapeutic Efficacy

In an embodiment, the present disclosure relates to methods of determining therapeutic efficacy of MLPSC populations (e.g. DP).

In an example, the method for determining therapeutic efficacy of a population of culture expanded mesenchymal lineage precursor or stem cells (MLPSCs) comprises: (i) obtaining a population of MLPSCs; (ii) culturing the cells in a culture medium; and (iii) determining under culture conditions the level of IL-2RA inhibition, wherein the method determines therapeutic efficacy for graft versus host disease (GvHD) and, wherein at least 56% IL-2RA inhibition under culture conditions is indicative of therapeutic efficacy. In another example, IL-2RA inhibition ≥60% is indicative of therapeutic efficacy. In another example, IL-2RA inhibition between 56% and 60% is indicative of therapeutic efficacy.

In an example, therapeutically effective mesenchymal lineage precursor or stem cell populations are capable of inhibiting IL-2RA expression by CD3/CD28-activated PBMCs by at least 56% relative to a control. In another example, therapeutically effective mesenchymal lineage precursor or stem cell populations are capable of inhibiting IL-2RA expression by CD3/CD28-activated PBMCs by at least 60% relative to a control. In another example, therapeutically effective mesenchymal lineage precursor or stem cell populations are capable of inhibiting IL-2RA expression by CD3/CD28-activated PBMCs between 56% and 70% relative to a control.

In an example, the obtained population of MLPSCs is representative of a larger MLPSC population. For example, the obtained population of MLPSCs may be representative of a series of cryopreserved MLPSC populations such as a series of doses. For example, the doses may be originate from the same population and one or more of the doses is obtained and assessed for therapeutic efficacy according to methods disclosed herein. In this example, the obtained dose(s) are representative of the remaining doses originating from the same MLPSC population. In an example, the obtained population of MLPSCs is representative of a population of MLPSCs in 3D culture.

Performing the methods of determining therapeutic efficacy disclosed herein can satisfy release criteria for release of therapeutically effective cell compositions as DP. In an example, such culture expanded compositions are characterized by a threshold level of IL-2RA inhibition. In an example, the level of IL-2RA inhibition is between 56% and 60% under culture conditions. In another example, the level of IL-2RA inhibition is ≥60% under culture conditions.

### Method of Manufacturing Drug Product

In an example, the present disclosure relates to a method of manufacturing drug product which comprises a population of MLPSCs, the method comprising: acquiring a determination of whether a test population of MLPSCs inhibits IL-2RA at a predetermined level under culture conditions, and processing at least a portion of the test population of MLPSCs as a drug product if the test population of MLPSCs inhibits IL-2RA by at least the predetermined level under culture conditions, thereby manufacturing the drug product; or discarding at least a portion of the test population of MLPSCs if the population of mesenchymal stem cells inhibits IL-2RA by less than the predetermined level under culture conditions, wherein the predetermined level is 56%. In an example, the predetermined level is ≥60% under culture conditions. In an example, the predetermined level is ≥65% under culture conditions.

In an example, the test population is obtained from a population of MLPSCs in 3D culture. For example, the MLPCSs can be in a bioreactor culture. In an example, the test population is obtained from cryopreserved population of MLPSCs. In an example, the test population is representative of a larger population of MLPSCs such as multiple cryopreserved populations of MLPSCs. In an example, the multiple cryopreserved populations of MLPSCs have been culture expanded from the same intermediate population of MLPSCs.

In an example, drug product comprises a composition disclosed herein. In an example, the drug product comprise 2×10⁶ MLPSCs.

In an example, acquiring a determination of whether a test population of MLPSCs inhibits IL-2RA at a predetermined level under culture conditions comprises co-culturing a representative amount of the MLPSCs according to the methods disclosed herein.

In an example, the present disclosure provides methods of manufacturing MSC drug product, such methods include a first step of providing (e.g., culture expanding (e.g., in small scale or large scale cell culture) or manufacturing) or obtaining (e.g., receiving and/or purchasing from a third party (including a contractually related third party or a non-contractually-related (e.g., an independent) third party) a test MSC population (e.g., a sample of a test MSC population), a second step of acquiring (e.g., detecting, measuring, receiving, or obtaining) at least one value (e.g., 1, 2, 3 and/or 4) for an MSC parameter listed in Table A for the test MSC population, and a third step of processing at least a portion of the test MSC population (e.g., processing a portion of a manufacturing lot, culture, or run, an entire manufacturing lot, culture, or run, or multiple manufacturing lots, cultures, or runs) as MSC drug product (e.g., in a form or packaging intended for administration as described subsequently herein; optionally cryopreserved) if the at least one value for the test MSC population meets a reference criterion shown in Table A for the parameter, thereby manufacturing MSC drug product. In an example, the value(s) comprise parameter number 1. In another example, the value(s) comprise parameter number 2. In another example, the value(s) comprise parameter number 3. In another example, the value(s) comprise parameter number 4. In another example, the value(s) comprise parameter number 1 and 2. In another example, the value(s) comprise parameter number 1 and 3. In another example, the value(s) comprise parameter number 1, 2 and 4.

In an example, such methods comprise a second step which includes acquiring values for any combination of two or more MSC parameters listed in Table A, and the third step of such methods includes processing at least a portion of the test MSC population as MSC drug product if the values for the any combination of two or more MSC parameters meet the corresponding reference criterion shown in Table A for the parameters.

TABLE A

| Parameter | | |
|---|---|---|
| # | Description | Reference Criterion |
| 1 | IL-2RA inhibition under culture | ≥60% |
| | conditions | |
| 2 | CD29, CD54, CD73, CD90, CD102, | CD29+, CD54+, CD73+, CD90+, CD102+, |
| | CD105, CD106, CD166, MHC1 | CD105+, CD106+, CD166+, MHC1+ |
| 3 | CD45, CD105, CD166 | CD45−, CD105+, CD166+ |
| 4 | CD45, CD105, CD166 | <0.75% CD45+, >95% CD105+; >95% |
| | | CD166+ |

### Selecting Cells for Use in Treatment of Graft Versus Host Disease (GvHD)

In an embodiment the present disclosure encompasses selecting cells for use in treatment of GvHD. In an example, the MLPSC population is assessed for the requisite criteria and, when met, said population is selected for use in treatment of GvHD.

In an example, an MLPSC population is selected for use in treatment of GvHD if it inhibits IL-2RA by between at least 56 and 60% under culture conditions. In an example, the MLPSC population is selected for use in treatment of GvHD if it inhibits IL-2RA by ≥60% under culture conditions. In an example, the MLPSC population is selected for use in treatment of GvHD if it inhibits IL-2RA by ≥65% under culture conditions.

Selection processes are not particularly limited so long as they are able to select cell populations characterized by the relevant criteria such as % IL-2RA inhibition. In an example, a series of intermediate MLPSC populations are assessed for IL-2RA inhibition under culture conditions and those populations which inhibit IL-2RA inhibition by at least 60% under culture conditions are selected for further expansion. In an example, a series of intermediate MLPSC populations are assessed for IL-2RA inhibition under culture conditions and those populations which inhibit IL-2RA inhibition by at least 65% under culture conditions are selected for further expansion.

In an example, selected mesenchymal lineage precursor or stem cell populations are capable of inhibiting IL-2RA expression by CD3/CD28-activated PBMCs by at least 56% relative to a control. In another example, selected mesenchymal lineage precursor or stem cell populations are capable of inhibiting IL-2RA expression by CD3/CD28-activated PBMCs by at least 60% relative to a control. In another example, selected mesenchymal lineage precursor or stem cell populations are capable of inhibiting IL-2RA expression by CD3/CD28-activated PBMCs between 56% and 70% relative to a control.

In an example, CD3/CD28-activated PBMCs express at least 10.00 pg/mL IL-2RA. In an example, CD3/CD28-activated PBMCs express at least 11.00 pg/mL IL-2RA. In an example, CD3/CD28-activated PBMCs express at least 12.00 pg/mL IL-2RA. In an example, CD3/CD28-activated PBMCs express at least 13,000 pg/mL IL-2RA. In an example, CD3/CD28-activated PBMCs express at least 12,811 pg/mL IL-2RA.

In an example, the obtained population of MLPSCs is representative of a larger MLPSC population. For example, the obtained population of MLPSCs may be representative of a series of cryopreserved MLPSC populations such as a series of doses. For example, the doses may be originate from the same population and one or more of the doses is obtained and assessed for therapeutic efficacy according to methods disclosed herein. In this example, the obtained dose(s) are representative of the remaining doses originating from the same MLPSC population. In an example, the obtained population of MLPSCs is representative of a population of MLPSCs in 3D culture. In these examples, cells from an alternative population of MLPSCs can be selected based on an assessment of a representative test population of MLPSCs.

In an example, selected cell populations are cryopreserved to allow administration at a later stage.

In another example, the present disclosure relates to compositions comprising population(s) of selected MLPSCs. In an example, the composition comprises a population of culture expanded MLPSCs, wherein the population of MLPSCs are selected based on a predetermined level of IL-2RA inhibition under culture conditions, wherein the predetermined level of IL-2RA inhibition is at least 56%. In another example, the predetermined level of IL-2RA inhibition is ≥60%. In another example, the MLPSCs are also selected based on expression of one or more or all of CD29, CD54, CD73, CD90, CD102, CD105, CD106, CD166, MHC1.

In an example, MLPSCs populations comprising compositions of the disclosure are selected based on one or more parameters described in Table A.

### Determining the Amount of IL-2RA Levels

The present disclosure contemplates any form of assay, including, for example, Western blot, enzyme-linked immunosorbent assay (ELISA), fluorescence-linked immunosorbent assay (FLISA), competition assay, radioimmunoassay, lateral flow immunoassay, flow-through immunoassay, electrochemiluminescent assay, nephelometric-based assays, turbidometric-based assay, fluorescence activated cell sorting (FACS)-based assays for determining the level of IL-2RA.

Following co-culture of MLPSCs and T cells, the cells can be collected and lysed using well-known methods in the art. The cell lysates can then be assayed for the presence of IL-2RA by, for example, ELISA or FLISA. Alternatively, the level of IL-2RA expression may be determined by assaying intact cells by, for example, flow cytometry.

In an example, the inhibition of IL-2RA expression is measured by comparing the level of IL-2RA expression of a population of cells comprising T cells to the level of IL-2RA of a population of cells following co-culture of a population of cells comprising T cells and a population of cells comprising mesenchymal lineage precursor or stem cells, and the difference expressed as “percentage inhibition”.

The assays described above are readily modified to use chemiluminescence or electrochemiluminescence as the basis for detection.

As will be apparent to the skilled person, other detection methods based on an immunosorbent assay are useful in the performance of the present disclosure. For example, an immunosorbent method based on the description above using a radiolabel for detection, or a gold label (e.g., colloidal gold) for detection, or a liposome, for example, encapsulating NAD+ for detection or an acridinium linked immunosorbent assay.

In some examples of the disclosure, the level of IL-2RA is determined using a surface plasmon resonance detector (e.g., BIAcore™, GE Healthcare, Piscataway, N.J.), a flow through device (e.g., as described in U.S. Pat. No. 7,205,159), a micro- or nano-immunoassay device (e.g., as described in U.S. Pat. No. 7,271,007), a lateral flow device (e.g., as described in US publication 20040228761 or US publication 20040265926), a fluorescence polarization immunoassay (FPIA, e.g., as described in U.S. Pat. No. 4,593,089 or U.S. Pat. No. 4,751,190), or an immunoturbidimetric assay (e.g., as described in U.S. Pat. No. 5,571,728 or U.S. Pat. No. 6,248,597).

### Compositions

The present disclosure encompasses compositions comprising a population of MLPSCs selected according to methods disclosed herein. In example, compositions of the present disclosure comprise a therapeutically effective amount of MLPSCs, wherein therapeutic efficacy is determined based on a method disclosed herein. In an example, the composition is a drug product manufactured according to the present disclosure, thereby having a predetermined level of IL-2Rα inhibition.

In certain examples, compositions of the disclosure comprise a pharmaceutically acceptable carrier and/or excipient.

The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. For example, such a media composition does not induce any adverse effects in a subject to whom it is administered.

Exemplary carriers and excipients do not adversely affect the viability of a cell and/or the ability of a cell to reduce, prevent or delay metabolic syndrome and/or obesity.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

In an example, a composition of the disclosure comprises between 1.5 million and 3 million cells/kg. In an example, a composition of the disclosure comprises 2 million cells/kg.

In an example, the composition comprises greater than 5.00×10⁶ viable cells/mL. In another example, the composition comprises greater than 5.50×10⁶ viable cells/mL. In another example, the composition comprises greater than 6.00×10⁶ viable cells/mL. In another example, the composition comprises greater than 6.50×10⁶ viable cells/mL. In another example, the composition comprises greater than 6.68×10⁶ viable cells/mL.

Compositions of the disclosure may be cryopreserved. Cryopreservation of mesenchymal lineage precursor or stem cells can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cryopreservation solution may comprise a sterile, non-pyrogenic isotonic solution such as, for example, PlasmaLyte A™. 100 mL of PlasmaLyte ATM contains 526 mg of sodium chloride, USP (NaCl); 502 mg of sodium gluconate (C₆H₁₁NaO₇); 368 mg of sodium acetate trihydrate, USP (C₂H₃NaO₂·3H₂O); 37 mg of potassium chloride, USP (KCl); and 30 mg of magnesium chloride, USP (MgCl₂·6H₂O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, αMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity. Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises about 5% human serum albumin (HSA) and about 10% DMSO. The cryopreservation solution may further comprise one or more of methycellulose, polyvinyl pyrrolidone (PVP) and trehalose.

In one embodiment, cells are suspended in 42.5% Profreeze™/50% αMEM/7.5% DMSO and cooled in a controlled-rate freezer.

The cryopreserved composition may be thawed and administered directly to the subject or added to another solution, for example, comprising HA. Alternatively, the cryopreserved composition may be thawed and the mesenchymal lineage precursor or stem cells resuspended in an alternate carrier prior to administration.

In an example, cellular compositions of the disclosure can comprise Plasma-Lyte A, dimethyl sulfoxide (DMSO) and human serum albumin (HSA). For example, compositions of the disclosure may comprise Plasma-Lyte A (70%), DMSO (10%), HSA (25%) solution, the HSA solution comprising 5% HSA and 15% buffer.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

The following specific examples are to be construed as merely illustrative, and not limitative of the remainder of the disclosure in any way whatsoever. Without further elaboration, it is believed that one skilled in the art can, based on the description herein, utilize the present invention to its fullest extent.

### EXAMPLES

### Example 1: Clinical Trial

241 children with steroid refractory acute graft versus host disease (aGvHD) who failed one or more additional therapies received mesenchymal stem cell (MSC) drug product (DP) as salvage therapy. Children with Grade D disease accounted for approximately 50% of patients referred for MSC as salvage therapy due to the most refractory nature of this stage of disease to all treatment modalities and its greatest mortality. The subjects in this study had failed steroid therapy as well as multiple salvage therapeutic interventions. This group of subjects provided an assessment of the effectiveness of DP in a patient population that had not previously responded to other conventional therapies. There was a high rate of overall response (OR) at Day 28 and overall survival (OS) at Day 100 across all grades of aGVHD, particularly for subjects with Grade D disease. These data highlighted the effectiveness of DP in a highly refractory patient population.

### Example 2: IL-2Rα Inhibition

Inhibitory effects on activated T cells is thought to reflect a key mechanism of action by which MSC DP has a therapeutic effect in aGVHD. An IL-2Rα potency assay has been developed to provide a functional measurement of the ability of MSC DP to inhibit IL-2Rα expression by activated T cells in vitro.

Day 100 OS and % IL-2Rα inhibition was assessed for each MSC DP administered to the 241 children with steroid refractory acute graft versus host disease (aGvHD). A sensitivity analysis was then performed to evaluate whether a threshold for minimum % IL-2Rα inhibition could be identified to explain the greater survival conferred by certain DP. The sensitivity analysis evaluated survival of children who received DP with mean % IL-2Rα inhibition above or below thresholds of 66% to 50%. Results are shown in Table 4.

TABLE 4
Analysis of Day 100 Overall Survival by Thresholds for
Mean % IL-2Rα Inhibition of Drug Product Received

| | | | Day 100 | | |
|---|---|---|---|---|---|
| IL-2Rα % Inhibition | | | Overall | | Chi-Sq |
| Threshold | N (%) | | Survival | | p-value |
| 54% | | | | | |
| At or above | 228 | (95%) | 152 | (67%) | |
| Below | 13 | (5%) | 7 | (54%) | 0.3426 |
| 55% | | | | | |
| At or above | 220 | (91%) | 149 | (68%) | |
| Below | 21 | (9%) | 10 | (48%) | 0.0631 |
| 56% | | | | | |
| At or above | 217 | (90%) | 148 | (68%) | |
| Below | 24 | (10%) | 11 | (46%) | 0.0282 |
| 57% | | | | | |
| At or above | 214 | (89%) | 147 | (69%) | |
| Below | 27 | (11%) | 12 | (44%) | 0.0122 |
| 58% | | | | | |
| At or above | 209 | (87%) | 144 | (69%) | |
| Below | 32 | (13%) | 15 | (47%) | 0.0143 |
| 59% | | | | | |
| At or above | 206 | (85%) | 143 | (69%) | |
| Below | 35 | (15%) | 16 | (46%) | 0.0062 |
| 60% | | | | | |
| At or above | 202 | (84%) | 139 | (69%) | |
| Below | 39 | (16%) | 20 | (51%) | 0.0344 |
| 61% | | | | | |
| At or above | 201 | (83%) | 138 | (69%) | |
| Below | 40 | (17%) | 21 | (53%) | 0.0489 |
| 62% | | | | | |
| At or above | 194 | (81%) | 133 | (69%) | |
| Below | 47 | (19%) | 26 | (55%) | 0.0857 |
| 63% | | | | | |
| At or above | 184 | (76%) | 127 | (69%) | |
| Below | 57 | (24%) | 32 | (56%) | 0.0729 |
| 64% | | | | | |
| At or above | 178 | (74%) | 122 | (69%) | |
| Below | 63 | (26%) | 37 | (59%) | 0.1579 |
| 65% | | | | | |
| At or above | 168 | (70%) | 114 | (68%) | |
| Below | 73 | (30%) | 45 | (62%) | 0.3495 |
| 66% | | | | | |
| At or above | 157 | (65%) | 106 | (68%) | |
| Below | 84 | (35%) | 53 | (63%) | 0.4901 |

Table 4 shows Day 100 survival outcomes were in the 67%-69% range for children who received DP lots with mean % IL-2Rα inhibition above any of the thresholds between 54%-66%. In contrast, Day 100 survival progressively decreased in a linear fashion in children who received DP lots with mean % IL-2Rα inhibition below 60%. Accordingly, the present inventors have identified that a threshold of ≥60% IL-2Rα inhibition would result in release of DP lots that confer a significant survival benefit in this disease. The present inventors findings therefore represent a significant advance in DP manufacture, in particular in the context of DP for treatment of T-cell mediated disorders such as GvHD.

### Example 3: Further Analysis

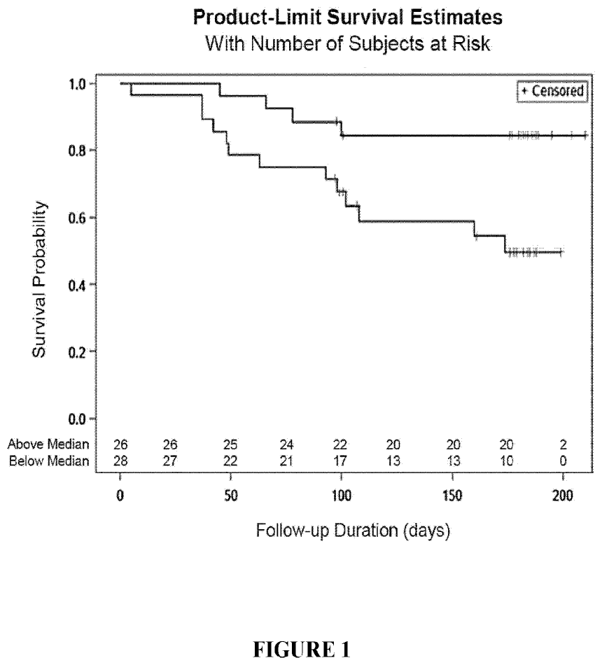

As shown in FIG. 1, there was a positive association between % IL-2Rα inhibition and Day 180 survival (85% Day 180 OS >median vs. 54% Day 180 OS ≤median, p=0.01). This survival benefit was preceded by a greater duration of the Day 28 OR among responders who received product with higher % IL-2Rα inhibition. The relationship between greater survival and mean % IL-2Rα inhibition >median vs. ≤median was most evident in patients with the most severe form of the disease and at highest risk for death:
a. Minnesota high risk (Day 180 OS 89% vs 50%, p=0.01);
b. MAGIC Algorithm Probability (MAP) ≥0.29 (Day 180 OS 100% vs. 17%, p=0.003);
c. IBMTR Grade D disease (Day 180 OS 91% vs. 50%, p=0.03).

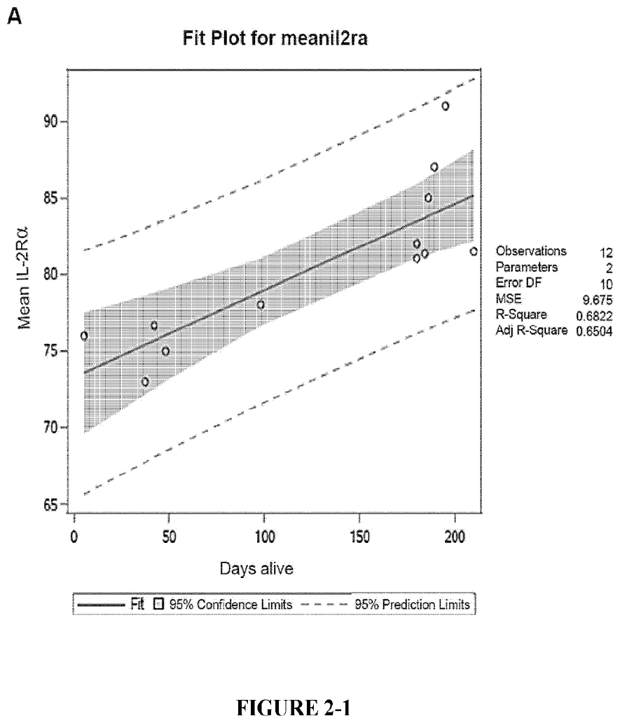

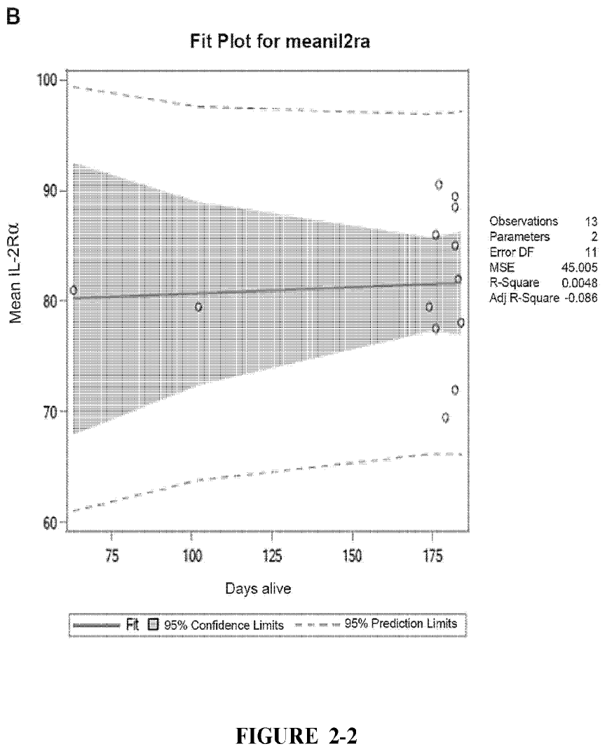

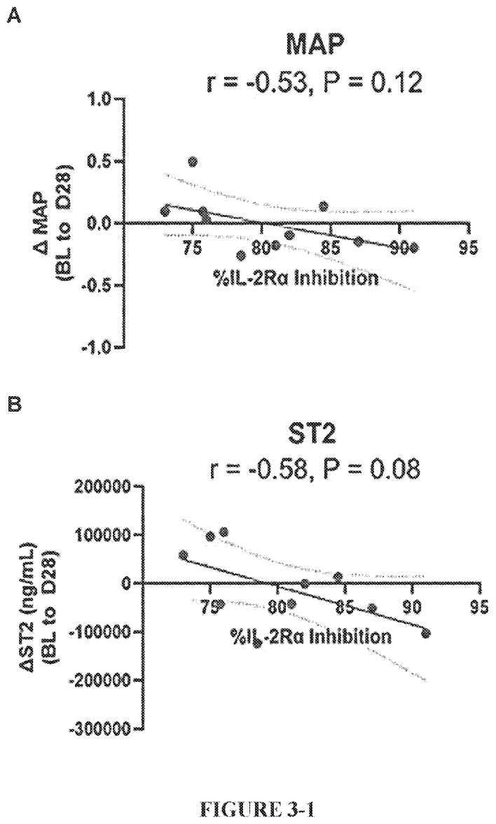

As shown in FIG. 2, in MAP high risk patients (MAP≥0.29), there was a significant correlation between progressively higher mean product % inhibition of IL-2Rα and increased days alive through 180 days (Pearson correlation coefficient=0.81, p=0.0013) (left figure). The lack of correlation between product potency and days alive in low-risk patients is likely reflective of all drug product being of sufficient potency to achieve survival in low-risk patients (right figure).

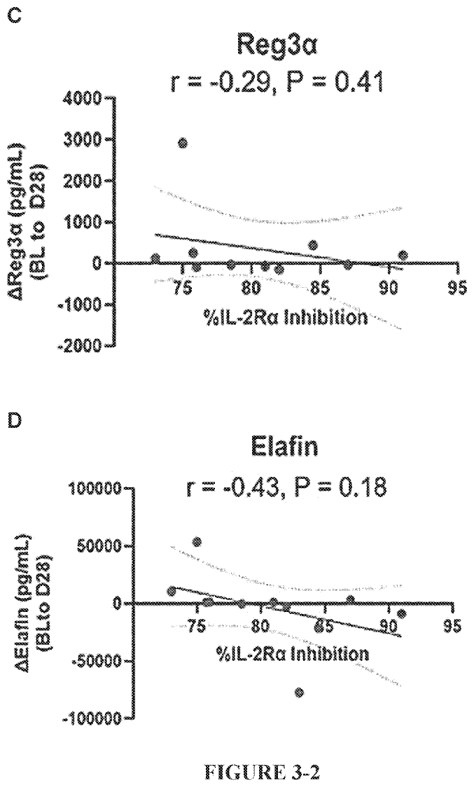

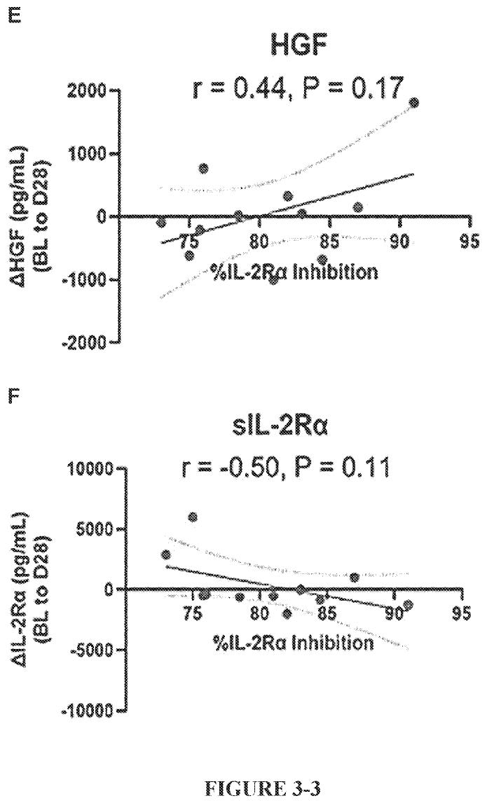

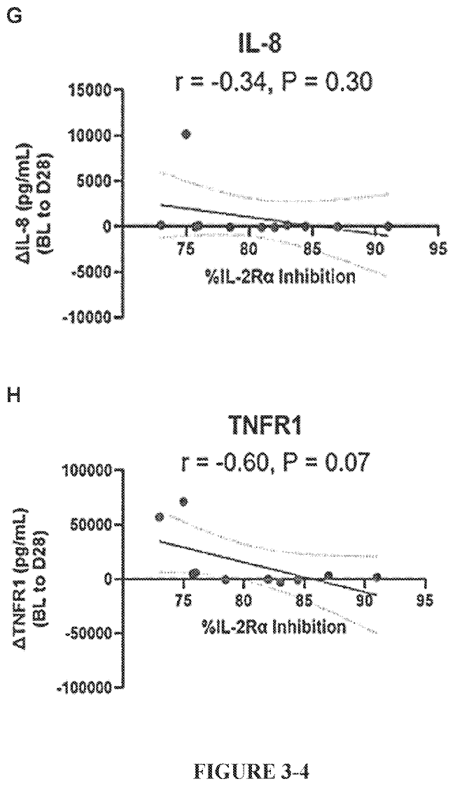

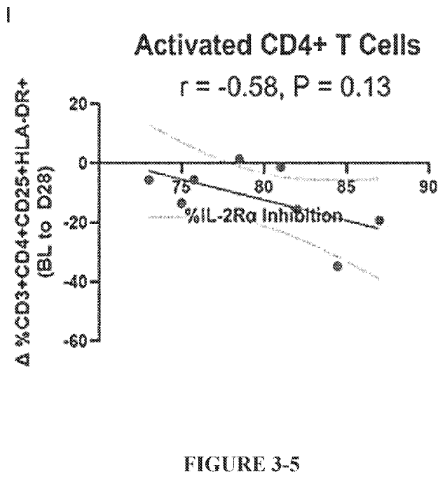

As shown in FIG. 3, among all children with high MAP scores (≥0.29) there was a linear negative correlation between progressively increasing IL-2Rα inhibition by MSC lots and reduction in various inflammatory biomarkers through 28 days.

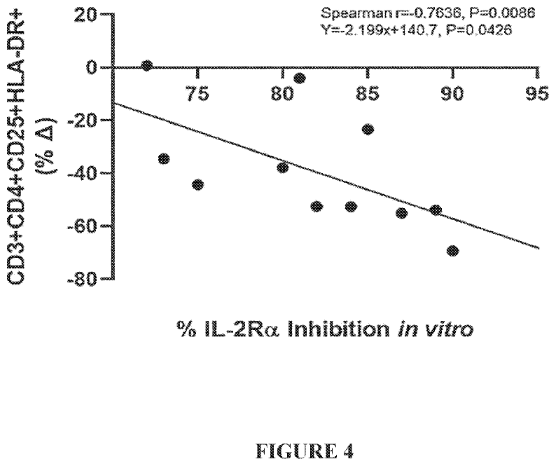

As shown in FIG. 4, for patients treated with a single lot of DP, potency significantly correlated with a decrease in the proportion of activated T cells in vivo between baseline and Day 28, as measured by T cells expressing CD3+CD4+CD25+HLA-DR+ phenotype.

### In Summary:

There was a significant relationship between % IL-2Rα inhibition in vitro and reduction in circulating levels of activated T cells from Screening to Day 28 (p=0.009).

Increasing product potency correlated with progressively decreasing inflammatory biomarkers in vivo and greater survival.

Survival benefit was most evident in those children with the most severe form of the disease, highest baseline levels of inflammatory biomarkers and at highest risk for death.

In patients with the greatest disease severity (Grade D) who received drug product with mean % IL-2Rα inhibition >median, Day 180 survival was significantly higher compared to those who received product with mean % IL-2Rα inhibition ≤median (91% vs. 50%, p=0.03).

Even the 50% survival achieved in those with Grade D SR-aGVHD who received product with mean % IL-2Rα inhibition ≤median is markedly higher than that seen with other treatment options where Day 180 survival has remained low at 12%-23% over the past two decades with best available treatment.

Accordingly, the IL-2Rα inhibition assay is a direct measure of drug products ability to inhibit T cell activation in vitro and correlates with drug products in vivo bioactivity as indicated by improved survival outcomes and reductions in circulating levels of inflammatory biomarkers and activated T cells.

### Example 4: Measurement of IL-2Rα Inhibition

Ce-MSC (either intermediate population or drug product) are co-cultured with un-stimulated or stimulated (CD3; CD28 antibodies) peripheral blood mononuclear cells (PBMC) as shown below. These conditions are representative of a ratio of approximately 1 stem cell:5 PMBC:

| | 1 | 2 | 3 | 4 | 5 | 6 |
|---|---|---|---|---|---|---|
| A | | | | | Sample 1:_{——}_{——}_{——} | |
| B | | 500 μl unstimulated | | 500 μl stimulated | Co-Culture - | |
| | | PBMC + 500 μl | | PBMC + 500 μl | 500 μl stimulated PBMC + | |
| | | Co-Culture media | | Co-Culture media | 500 μl ceMSCs | |
| C | | | | | □ N/A | |
| | | | | | Sample 2:_{——}_{——}_{——} | |
| D | | | | | Co-Culture - 500 μl | |
| | | | | | stimulated PBMC + | |
| | | | | | 500 μl ceMSCs | |

Plates are incubated at 37° C.+/−2° C., 5+/−2% CO₂ for 60-84 hours before cells are harvested, lysed and assessed for IL-2RA expression by activated PBMCs using a commercially available ELISA kit, according to the manufacturer's instructions (R&D systems).

The inhibition of IL-2RA expression on PBMCs by MSC lots (IL-2Rα inhibition assay) is a quantitative bioactivity assay that provides a direct measurement of the inhibitory effects of a cell population (e.g. final product). Measurement of this bioactivity enables evaluation of the populations inhibitory effect on activated T cells in vitro.

### Example 5: Relationship Between PBMC Stimulation and IL-2Rα Inhibition

The IL-2RA inhibition assay involves culturing MSCs for 72 hours under standard culture conditions, then a further 72 hour co-culture with PBMCs. IL-2RA inhibition is measured on day 6 of culture. IL-2Rα inhibition is a critical quality attribute that measures the effect of MSCs on the key mechanism by which the immune system mediates disease severity in SR-aGVHD, namely alloreactive T cell activation, proliferation and inflammatory cytokine production.

The degree of PBMC stimulation and subsequent inflammatory cytokine release when measuring IL-2RA inhibition is a critical component in the ability to induce MSC lot release of immunomodulatory factors necessary for IL-2RA inhibitory activity, such as IDO-1, PGE2 and PD-L1. To assess the minimum level of PBMC stimulation by anti-CD3 and anti-CD28 antibodies necessary to result in reliable measurement of lot potency, regression analyses was performed to assess the relationship between IL-2RA produced by stimulated PBMC and degree of induced suppressive activity by MSC lots.

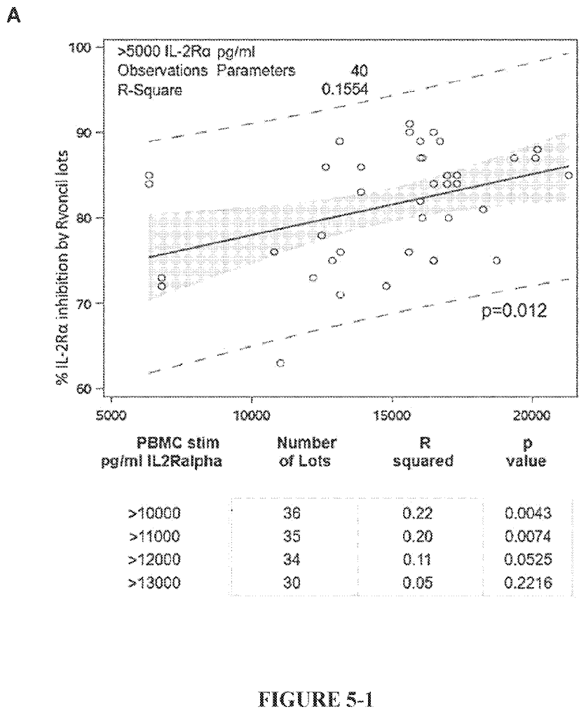

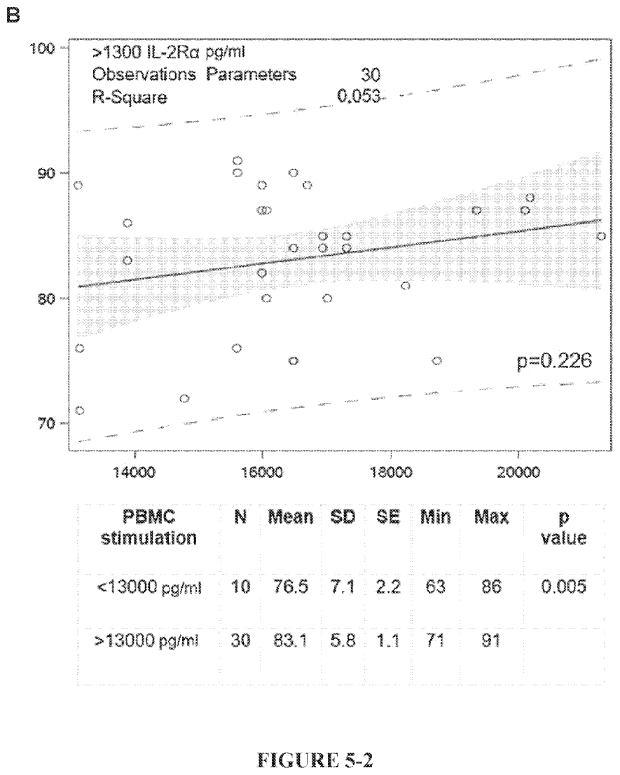

As shown in FIG. 5, there was an observed correlation between the amount of IL-2RA produced by stimulated PBMC and % inhibition of IL-2RA across all 40 lots tested (p=0.012). This association remained significant at post-stimulation PBMC IL-2RA expression thresholds of 10000, 11000 and 12000 pg/ml, but was no longer evident when the amount of IL-2RA produced by stimulated PBMC exceeded 13000 pg/ml. Mean IL-2RA inhibition by the 30 lots tested with PBMC producing >13000 pg/ml IL-2RA was 83% (range 71-91%) compared with 77% inhibition (range 63-86) for 10 lots tested with PBMC producing <13000 IL-2RA (p=0.005).

These results indicate that IL-2RA production of 12000-13000 pg/ml reflects the minimum threshold of PBMC stimulation necessary to ensure that there is adequate activation of MSC lots to induce maximum IL-2RA inhibitory activity. While the mean IL-2RA inhibitory activity for the lots tested with PBMC producing <13000 IL-2RA was apparently lower than for those tested with PBMC producing >13000 pg/ml IL-2RA, these data indicate that potency of MSC lots tested with inadequately stimulated PBMC is not reliable and likely underestimates the true potency values for these lots.

### Example 6: Survivors in GvHD Clinical Trial Received MSCs with Higher Mean % IL2R Inhibition

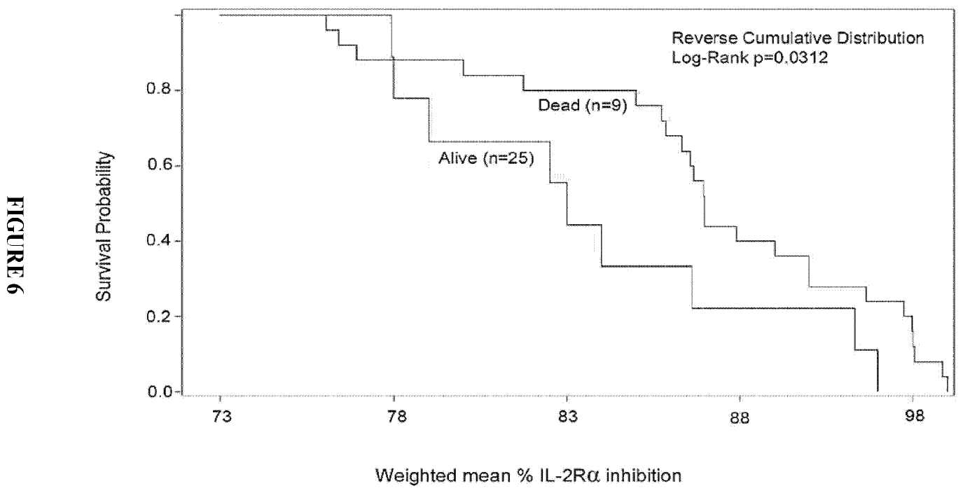

Next, Reverse Cumulative Distribution curves were used to assess the relationship between product potency and clinical outcomes in those patients (n=34) who received lots with potency values measured with adequately stimulated PBMC (n=30 lots, >12811 pg/ml IL-2RA) (FIG. 6).

As can be seen in FIG. 6, there was a significant relationship between weighted mean value of % IL-2RA inhibition of lots received and day 100 survivorship by Reverse Cumulative Distribution curves (p=0.03). Since mean IL-2RA inhibition was 83.1%±5.8% for these 30 lots, 65.7% was used as the minimum threshold IL-2RA inhibition required for lot release by the IL-2RA inhibition assay with adequately stimulated PBMC (≥12811 pg/ml IL-2RA).

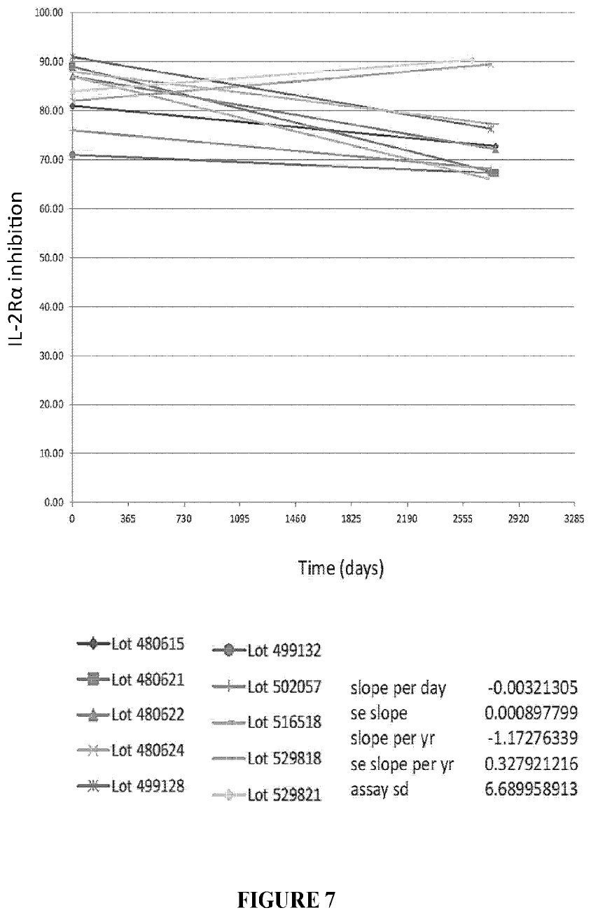

Clinical lots tested in 2015 with the IL-2RA inhibition assay with adequately stimulated PBMC (≥12811 pg/ml IL-2RA) were retested in 2023 to evaluate whether potency was maintained on cryostorage. As seen in FIG. 7, IL-2Rα inhibition measured at two timepoints with the same clinical lot, first tested in 2015 and subsequently re-tested in 2023, demonstrated a high degree of clinical lot stability for at least 8 years in cryostorage.

### Example 7: Accurate Measurement of % Inhibition of IL-2Rα Inhibition Requires Adequate PBMC Stimulation and Induced Levels of IL-2Rα Expression

The slope of degradation for IL-2Rα inhibition value is 1.2% per year, indicating that MSC DP lots remain highly stable over time with respect to IL-2Rα inhibition. Comparing the regression lines with actual assay values provides a highly sensitive, real-world test of IL-2Rα inhibition assay precision, as assessed in the 10 lots for which adequately stimulated PBMCs were used at both time points. The ability of the linear stability modeling using a common slope to predict actual test values was used to calculate the assay standard deviation based on the residuals in the linear regression. This calculation indicates a standard deviation for the IL-2Rα inhibition assay of less than 7%, indicating sufficient precision for use of this assay to measure a critical quality attribute.

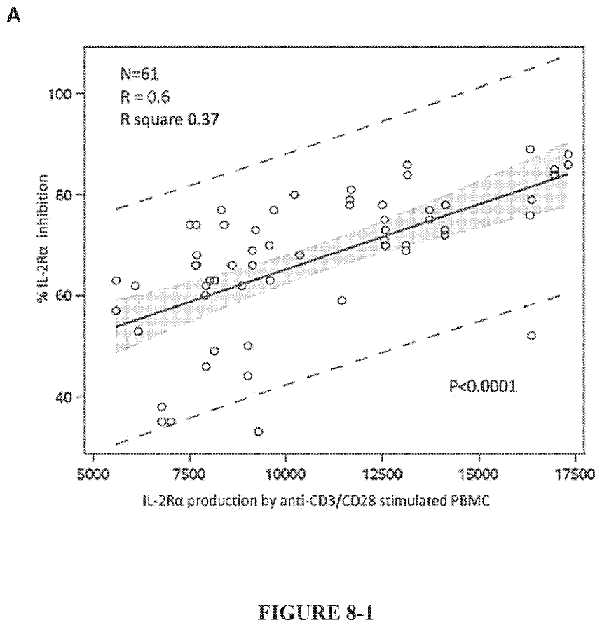

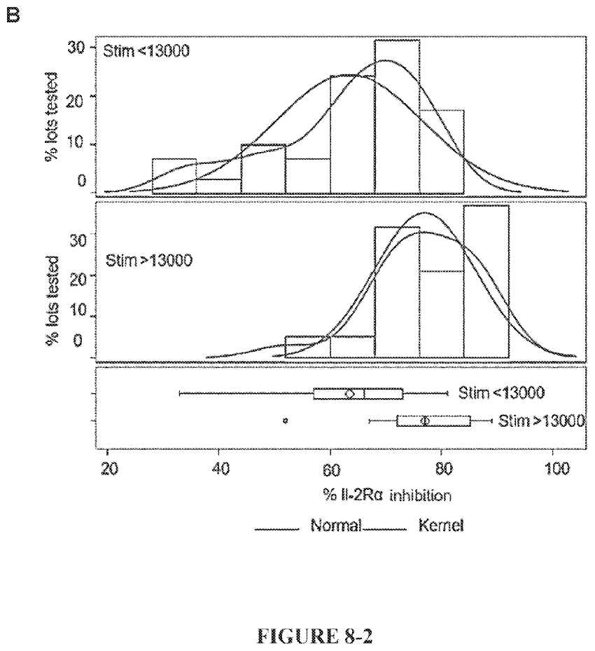

To confirm the relationship observed in clinical lots between adequate PBMC stimulation, as measured by IL-2Rα production, and MSC lot potency, a regression analysis was performed based on these same parameters for 61 lots. As can be seen in FIG. 8 (left box), in these 61 additional MSC lots there was also a significant linear correlation between PBMC production of IL-2RA following anti-CD3/CD28 stimulation and degree of % IL-2R alpha inhibition (p<0.0001). This is consistent with IL2R production by activated PBMC being a biomarker for inflammatory cytokine secretion, such as IFN gamma, TNF alpha and IL-1, which are responsible for induction of anti-inflammatory factors secreted by Ryoncil lots, including IDO-1, PGE2 and PD-L1.

As with the earlier clinical lots, correlation between PBMC production of IL-2RA and IL-2RA inhibition by co-cultured MSC lots was only observed up to stimulated PBMC producing 12000-13000 pg/ml IL-2RA, indicating that induction of MSC suppressive potency was maximal above these levels of PBMC stimulation. While 95% of lots tested with PBMC stimulated to produce >13000 pg/ml IL-2RA showed >60% inhibition of IL-2RA, only 70% of lots tested with PBMC stimulated suboptimally to produce lesser amounts of IL2R alpha achieved this level of inhibition, FIG. 8 (right box).

These results confirm that accurate measurement of MSC lot potency for inhibition of IL-2RA requires optimal PBMC stimulation and inflammatory cytokine secretion, and these can be achieved by quality control and strict acceptance criteria for PBMC stimulation with anti-CD3/CD28 antibodies.

### Example 8: Acceptance Criteria for IL-2RA Inhibition Assays

In view of the data above, the value of 65.7% represents the minimum threshold of IL-2RA inhibition met by 95% of all lots used in clinical trials as measured by the IL-2RA inhibition assay when PBMC stimulation achieved IL-2RA levels ≥12811 pg/ml.

### Example 9: IL2Ra Inhibition Assay Protocol and Validation Procedure

The expression of Interleukin-2 receptor a (IL-2Rα/IL-2RA) on the cell surface is an early marker T-cell activation. A major function of IL-2/IL-2Rα is to promote proliferation of CD4+ (helper) and CD8+ (cytotoxic) T-cells. Inhibition of IL-2Rα expression results in suppression T-cell proliferation and further expansion of the immune response. ceMSCs are utilized for performing the IL-2Rα inhibition assay as follows:

MSCs (2.00×10⁵ cells/well) are co-cultured with mouse anti-human CD3/CD28 stimulated PBMCs (1.00×10⁶ cells/well) and incubated for 72±2 hours at 37° C.±2° C., 5%±2% CO₂.

Unstimulated (without CD3/CD28) and stimulated PBMC (with CD3/CD28, but without ceMSCs) are included as negative and positive controls, respectively.

At the end of the incubation period, cells are collected for lysate preparation. The cell lysates are then aliquoted in sterile tubes for storage at ≤−60° C. up to 29 days until use in IL-2Rα ELISA.

The IL-2Rα levels in lysates of unstimulated and stimulated PBMC alone as well as co-culture are measured at one dilution in triplicate using Quantikine Human CD25/IL-2 Rα ELISA kit.

A standard curve constructed using a four-parameter logistic (4-PL) curve fit is used to determine IL-2Rα levels in controls and samples. Final ceMSC potency is evaluated by the % inhibition of IL-2Rα levels on CD3/CD28-stimulated PBMC by ceMSC relative to the control CD3/CD28-stimulated PBMC cultured without ceMSC.

### Validation

The innate variability introduced in the potency testing of cellular materials against a secondary reagent cell line requires additional measures be considered. To address this inherent assay variability, it was decided to utilize a normalization factor that corrects for the donor-to-donor variability inherent in the assay's PBMC reagent.

Investigations into the variability of the IL-2Rα Inhibition assay showed the composite effect of the PBMC donor in the mechanism of suppression and the PBMC variability in response to anti-CD3/anti-CD28 stimulation are important parameters.

Accordingly, appropriate controls will include the screening of PBMC lots to confirm the reproducibility of the PBMCs to stimulate above 12,811 pg/mL of IL-2Rα and verification of the acceptability of the test PBMC to meet the ceMSC reference cell line standard range in the average of testing at least 4 vials (i.e., 4 executions).

ceMSC reference cell lines can be generated by the same 3 lots of ceMSC (generated from 3 bone marrow donors) used in the IL-2Rα Inhibition assay validation and the acceptance range will be calculated based on the values generated in the validation. The ceMSC reference cell lines will then be utilized to qualify new PBMC lots for use in the IL-2Rα inhibition assay. The ceMSC reference cell lines will be qualified and utilized to ensure the normalization factor is accurately generated for each PBMC lot and there is continued conformity of the assay performance over time.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from U.S. 63/444,198 filed 8 Feb. 2023, and U.S. 63/445,588 filed 14 Feb. 2023, the disclosures of which are incorporated herein by reference.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A method of selecting a cell population for use in treatment of graft versus host disease (GvHD), the method comprising: (i) obtaining a population of mesenchymal lineage precursor or stem cells (MLPSCs); (ii) culturing the MLPSC population in a culture medium; (iii) determining under culture conditions the level of IL-2RA inhibition; and, (iv) selecting for use in therapy an MLPSC population that inhibits IL-2RA by at least 56% under culture conditions.

2. The method of claim 1, which comprises selecting an MLPSC population for use in therapy which inhibits IL-2RA by at least 60% under culture conditions.

3. The method according to claim 1 or claim 2, further comprising cryopreserving the selected cells.

4. A method for determining therapeutic efficacy of a population of culture expanded mesenchymal lineage precursor or stem cells (MLPSCs): (i) obtaining a population of MLPSCs; (ii) culturing the cells in a culture medium; and (iii) determining under culture conditions the level of IL-2RA inhibition, wherein the method determines therapeutic efficacy for graft versus host disease (GvHD) and, wherein at least 56% IL-2RA inhibition under culture conditions is indicative of therapeutic efficacy.

5. The method of claim 4, wherein ≥60% IL-2RA inhibition under culture conditions is indicative of therapeutic efficacy.

6. A method of manufacturing drug product which comprises a population of MLPSCs, the method comprising: acquiring a determination of whether a test population of MLPSCs inhibits IL-2RA at a predetermined level under culture conditions, and processing at least a portion of the test population of MLPSCs as a drug product if the test population of MLPSCs inhibits IL-2RA by at least the predetermined level under culture conditions, thereby manufacturing the drug product; or discarding at least a portion of the test population of MLPSCs if the population of mesenchymal stem cells inhibits IL-2RA by less than the predetermined level under culture conditions, wherein the predetermined level is 56%.

7. The method of claim 6, wherein the predetermined level ≥60% under culture conditions.

8. A method of treating a subject with graft versus host disease (GvHD), the method comprising administering to a subject in need thereof a composition comprising a culture expanded population of mesenchymal lineage precursor or stem cells (MLPSCs), wherein the MLPSCs inhibit IL-2RA by at least 56% under culture conditions.

9. The method of claim 8, wherein the culture expanded MLPSCs which comprise the administered composition inhibit IL-2RA by ≥60% under culture conditions.

10. The method according to any one of claims 1 to 5, 8 and 9, wherein the GvHD is acute GvHD.

11. The method according to any one of claims 1 to 5, 8 and 9, wherein the GvHD is chronic GvHD.

12. The method according to any one of claims 1 to 5 and 8 to 11, wherein the GvHD is pediatric GvHD.

13. The method according to any one of claims 1 to 5 and 8 to 12, wherein the GvHD is refractory to steroid therapy.

14. The method according to any one of claims 1 to 5 and 8 to 13, wherein the GvHD is Grade D GvHD.

15. The method according to any one of claims 8 to 14, wherein treatment increases 100 day survival to greater than 60%, greater than 70%.

16. The method according to any one of claims 1 to 15, wherein the MLPSCs are mesenchymal stem cells.

17. A composition comprising a population of culture expanded MLPSCs, wherein the population of MLPSCs are selected based on a predetermined level of IL-2RA inhibition under culture conditions, wherein the predetermined level of IL-2RA inhibition is at least 56%.

18. The composition of claim 17, wherein the predetermined level of IL-2RA inhibition is ≥60%.

19. The composition of claim 17 or 18, wherein the MLPSCs are mesenchymal stem cells.

20. The composition of claim 19, wherein the mesenchymal stem cells are also selected based on expression of one or more or all of CD29, CD54, CD73, CD90, CD102, CD105, CD106, CD166, MHC1.

21. The method according to any one of claims 1 to 16, or the composition according to any one of claims 17 to 20, wherein level of IL-2RA inhibition is ≥65%.

22. A method of manufacturing drug product which comprises a population of MLPSCs, the method comprising
(i) acquiring a determination of whether a test population of MLPSCs inhibits IL-2RA at a predetermined level under culture conditions, wherein step (i) comprises co-culturing the test population of MLPSCs with CD3/CD28 activated PBMCs; and determining the level of IL-2RA inhibition relative to a control population of CD3/CD28 activated PBMCs in the absence of MLPSCs; wherein the predetermined level of IL-2RA inhibition is at least 56%.

23. The method of claim 22, wherein the control population of CD3/CD28 activated PBMCs express at least 12,000 pg/ml IL-2RA.

24. The method of claim 22 or 23, wherein CD3/CD28 activation of PBMCs comprises stimulating PBMCs with an anti-CD3 antibody and an anti-CD28 antibody.

25. The method according to any one of claims 22 to 24, wherein the PBMCs are co-cultured with MLPSCs at a ratio of 5 PBMC:1 MPLSC.

26. The method according to claim 25, wherein 1×10⁶ PBMCs are co-cultured with 2×10⁵ MLPSCs.

27. The method according to any one of claims 22 to 26, wherein the PBMCs and MLPSCs are co-cultured for a period of about 72 hours.

28. The method according to any one of claims 22 to 27, wherein the level of IL-2RA inhibition is determined by:
(i) measuring IL-2RA levels in a test population of MLPSCs co-cultured with CD3/CD28-activated PBMCs, a positive control population of CD3/CD28-activated PBMCs in the absence of MLPSCs, and a negative control population of un-activated PBMCs in the absence of MLPSCs;
(ii) constructing a standard curve to determine IL-2RA levels in the positive control population, negative control population, and test population; and
(iii) calculating % inhibition of IL-2RA levels on the positive control population by the test population of MLPSCs co-cultured with CD3/CD28-activated PBMCs, relative to the positive control CD3/CD28-activated PBMC population.

29. The method of claim 28, wherein the IL-2RA levels are measured by enzyme-linked immunosorbent assay (ELISA).

30. The method of claim 29 or 29, wherein the standard curve is generated using a four-parameter logistic (4-PL) curve fit.
